(19) United States
(12) Patent Application Publication
(10) Pub. No.: US 2005/0153991 A1
(43) Pub. Date: Jul. 14, 2005

(54) Methods of treatment of chronic lymphocytic leukemia using roscovitine

(75) Inventors: Athos Gianella-Borradori, Dundee (GB); David Philip Lane, Dundee (GB); Paul Moss, Edgbaston (GB); Tatjana Stankovic, Edgbaston (GB); Simon Richard Green, Dundee (GB)

Correspondence Address: LAHIVE & COCKFIELD, LLP., 28 STATE STREET, BOSTON, MA, 02109, US

(73) Assignee: Cyclacel Limited, Dundee (GB)

(21) Appl. No.: 10/881,869

(22) Filed: Jun. 30, 2004

(30) Foreign Application Priority Data
Jun. 30, 2003 (GB) GB0315259.2

(51) Int. Cl. A61K 31/52

(52) U.S. Cl. 514/263.4

(57) ABSTRACT

The present invention relates to a method of treating a patient suffering from chronic lymphocytic leukemia (CLL) comprising administering a therapeutically effective amount of roscovitine or a pharmaceutically effective salt thereof.

### RELATED APPLICATIONS

This application is a continuation in part of Great Britain Application No. GB0315259.2, filed Jun. 30, 2003, the entire contents of which are hereby incorporated by reference.

### FIELD OF THE INVENTION

The present invention relates to the therapeutic uses of the compound 2-[(1-ethyl-2-hydroxyethyl)amino]-6-benzylamine-9-isopropylpurine and pharmaceutically acceptable salts thereof.

### BACKGROUND TO THE INVENTION

Cyclin-dependent kinases (CDKs) are serine/threonine kinases that play a crucial regulatory role in the cell cycle. CDKs regulate cell cycle progression by phosphorylation of various proteins involved in DNA replication and cell division, including transcription factors and tumour suppressor proteins.⁵ Certain CDKs also play a role in the regulation of RNA synthesis by their involvement in the phosphorylation of the carboxy terminal domain (CTD) of the largest subunit of RNA polymerase II (pol II). It is not surprising, therefore, that CDKs have become attractive therapeutic targets. Consequently, many new pharmacological agents capable of interfering with the activity of CDKs by competing for their ATP binding site are currently being tested in clinical trials.⁶

The prior art has described several compounds that are capable of regulating the cell cycle by virtue of inhibiting cyclin dependent kinases. These compounds include butyrolactone, flavopiridol and 2-(2-hydroxyethylamino)-6-benzylamino-9-methylpurine (olomoucin). Olomucin and related compounds have been shown to be inhibitors of cdc2. Cdc2 (also known as cdk1) is a catalytic sub-unit of a family of cyclin dependent kinases that are involved in cell cycle regulation.

These kinases comprise at least two sub-units, namely a catalytic sub-unit (of which cdc2 is the prototype) and a regulatory sub-unit (cyclin). The cdks are regulated by transitory association with a member of the cyclin family: cyclin A (cdc2, CDK2), cyclin B1-B3 (cdc2), cyclin C (CDK8), cycline D1-D3 (CDK2-CDK4-CDK5-CDK6), cyclin E (CDK2), cyclin H (CDK7).

Each of these complexes is involved in a phase of the cellular cycle. CDK activity is regulated by post-translatory modification, by transitory associations with other proteins and by modifications of their intra-cellular localization. The CDK regulators comprise activators (cyclins, CDK7/cyclin H, cdc25 phosphateses), the p9CKS and p15CDK-BP sub-units, and the inhibiting proteins (p16INK4A, p15INK4B, p21Cipl, p18, p27Kipl).

There is now considerable support in the literature for the hypothesis that CDKs and their regulatory proteins play a significant role in the development of human tumors. Thus, in numerous tumors a temporal abnormal expression of cyclin-dependent kinases, and a major de-regulation of protein inhibitors (mutations, deletions) has been observed.

Roscovitine has been demonstrated to be a potent inhibitor of cyclin dependent kinase enzymes, particularly CDK2. CDK inhibitors are understood to block passage of cells from the G1/S and the G2/M phase of the cell cycle. The pure R-enantiomer of Roscovitine, CYC202 (R-Roscovitine) has recently emerged as a potent inducer of apoptosis in a variety of tumour cells⁷ and is already in clinical trials to treat breast cancer and non-small cell lung cancer.⁶ Roscovitine has also been shown to be an inhibitor of retinoblastoma phosphorylation and therefore implicated as acting more potently on Rb positive tumors.

It has now been observed that roscovitine has therapeutic applications in the treatment of certain proliferative disorders that have to date been particularly difficult to treat.

### SUMMARY OF THE INVENTION

A first aspect of the invention relates to a method of treating a patient suffering from chronic lymphocytic leukemia (CLL) comprising administering a therapeutically effective amount of roscovitine or a pharmaceutically effective salt thereof.

A second aspect of the invention relates to a method of treating a patient suffering from an ATM-mutant chronic lymphocytic leukemia comprising administering a therapeutically effective amount of roscovitine or a pharmaceutically effective salt thereof.

A third aspect of the invention relates to a method of treating a patient suffering from a TP53 mutant chronic lymphocytic leukemia comprising administering a therapeutically effective amount of roscovitine or a pharmaceutically effective salt thereof.

A fourth aspect of the invention relates to a method of down regulating expression of an anti-apoptotic gene in B-cell chronic lymphocytic leukemia cells, the method comprising contacting the cells with roscovitine, or a pharmaceutically acceptable salt thereof.

A fifth aspect of the invention relates to a method of treating chronic lymphocytic leukemia in a subject, the method comprising administering roscovitine, or a pharmaceutically acceptable salt thereof, to the subject in an amount sufficient to down regulate the expression of an anti-apoptotic gene in the subject.

A sixth aspect of the invention relates to a method of down regulating expression of a DNA repair gene in B-cell chronic lymphocytic leukemia cells, the method comprising contacting the cells with roscovitine, or a pharmaceutically acceptable salt thereof.

A seventh aspect of the invention relates to a method of treating chronic lymphocytic leukemia in a subject, the method comprising administering roscovitine or a pharmaceutically acceptable salt thereof, to the subject in an amount sufficient to down regulate the expression of a DNA repair gene in the subject.

An eighth aspect of the invention relates to a method of down regulating expression of a gene involved in transcription regulation in B-cell chronic lymphocytic leukemia cells, the method comprising contacting the cells with roscovitine, or a pharmaceutically acceptable salt thereof.

A ninth aspect of the invention relates to a method of treating chronic lymphocytic leukemia in a subject, the method comprising administering roscovitine, or a pharmaceutically acceptable salt thereof, to the subject in an amount sufficient to down regulate a gene involved in the regulation of transcription in the subject.

A tenth aspect of the invention relates to a pharmaceutical composition for use in the treatment of chronic lymphocytic leukemia comprising roscovitine, or a pharmaceutically acceptable salt thereof, and a pharmaceutically acceptable carrier.

### BRIEF DESCRIPTION OF THE DRAWINGS

FIG. 1 shows the CD8+/CD4+ double positive immunophenotype of TPLL-1 cells (A) and HLA-B27 histogram (B).

FIG. 2 shows comparative genomic hybridization (CGH). Hybridization of tumour DNA was detected with FITC and the reference DNA hybridization with TRITC

FIG. 3 shows fluorescent in situ hybridization (FISH) analysis of c-myc in TPLL-1 cells.

FIG. 4 shows detection of three copies of c-myc by dual colour FISH.

FIG. 5 shows that incubation with roscovitine (CYC-202) for 5 h resulted in apoptosis of 96% of TPLL-1 cells, which was not inhibited by phorbol esters (10 nM TPA).

FIG. 6 shows the results of incubation for 18 h with 10 μM roscovitine.

FIG. 7 shows the results of testing TPLL-1 cells for cyclin A, B1, D1 and E expression.

FIG. 8 shows the expression of apoptotic inhibitor Bcl-2 in TPLL-1 cells.

FIG. 9 shows the effect of 10 μM roscovitine on TPLL-1 intracellular stress signal pathways.

FIG. 10 shows the results of immunoblotting with antibodies specific for phosphrylated by PI-3K-dependent manner sites of the Akt/PKB (Ser-473) and Raf-1 (Ser-338).

FIG. 11 shows cell viability over time of B-CLL tumours with no treatment, irradiation, or drugs. a) Dose-response of B-CLL tumours to CYC202 as shown by percentage of viable cells of 26 of 26 tumours without treatment, 24 of 26 tumours treated with 1 μg/ml CYC202, 19 of 26 treated with 2.5 μg/ml CYC202 and 26 of 26 treated with 5 μg/ml of CYC202. Cell viability and apoptosis were measured by annexin V assay; b) to d): B-CLLs separated by genotype: b) 15 ATM wild-type tumours with no treatment, or treated with 5 Grays of irradiation, 20 μM Fludarabine or 5 μg/ml CYC202. Cells analysed by Annexin V assay as in a); c) 7 ATM mutant tumours; d) 4 TP53-mutant tumours. Cell viability and apoptosis were measured by annexin V assay.

FIG. 12 shows: a) a comparison of the decrease in the percentage of viable B-CLL cells (all subtypes combined) over a 24-hour period when exposed to 5 μg/ml CYC202, 20 μM fludarabine, 5 Grays of irradiation, or no treatment; b) the decrease in viability in B-CLL cells by subtype when exposed to 5 μg/ml CYC202, 20 μM fludarabine, 5 Grays, or no treatment.

FIG. 13 shows a) the effect of incubation with 5 μg/ml CYC202 on the viability of normal B cells (solid line) and B-CLL cells (dashed line) as analysed with annexin V at 24, 48 and 72 hours; and b) in vitro response of normal cells vs B-CLL cells to CYC202 (51 g/ml). In normal peripheral B cells, loss of viability was less dramatic and the increase in apoptosis following treatment with CYC202 was less than 60% compared to over 80% for B-CLL cells.

FIG. 14 shows the effect of incubation of B-CLL cells with 5 μg/ml CYC202 on the expression of a) p53 and p21 proteins and b) cleavage of PARP1, procaspase-3 and procaspase-7. ATM wild-type cells were treated for 0, 1, 2, 3, 4, 5, 6 and 24 hours, ATM mutant cells for 0, 2, 10 and 18 hours and TP53 mutant cells for 0, 2, 4, 6, 8, 10 and 24 hours and proteins extracted and analysed by western blot for p53 and p21 expression. Actin was used as a loading control.

FIG. 15 shows: a) microarray analysis showing representative genes down-regulated in 5 B-CLL tumours treated with 5 μg/ml CYC202 for 4 hours compared to the same untreated tumours; b) confirmation by western blot of reduced expression of two anti-apoptotic proteins, Mcl-1 and Bcl-2, in all three B-CLL subtypes; c) absence of down-regulation of Mcl-1 and PARP1 cleavage in ATM wild-type B-CLL cells cultured for up to 24 hours; d) confirmation by western blot of reduced expression of PCNA in B-CLL cells; e) microarray analysis of B-CLLs before and after treatment with CYC202.

FIG. 16 shows: a) Down-regulation of phosphorylation of RNA pol II after CYC202 treatment of two ATM wild-type B-CLL tumours as shown by western blot with a phospho-specific antibody to Serine 2 of RNA pol II; b) down-regulation of total RNA pol II protein levels by 24 hours of CYC202 treatment.

### DETAILED DESCRIPTION

As mentioned above, the present invention relates to the use of roscovitine in the treatment of chronic lymphocytic leukemia (CLL).

Roscovitine or 2-[(1-ethyl-2-hydroxyethyl)amino]-6-benzylamine-9-isopropylpurine, is also described as 2-(1-D,L-hydroxymethylpropylamino)-6-benzylamine-9-isopropyl-purine. As used herein, the term “roscovitine” encompasses the resolved R and S enantiomers, mixtures thereof, and the racemate thereof.

As used herein, the term “CYC202” refers to the R enantiomer of roscovitine, namely, 2-(1-R-hydroxymethylpropylamino)-6-benzylamino-9-isopropylpurine, the structure of which is shown below.

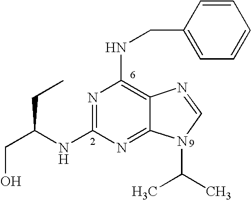

The in vitro activity of roscovitine is as follows:

| Kinase | IC₅₀ (μM) |
|---|---|
| Cdk1/cyclin B | 2.7 |
| Cdk2/cyclin A | 0.7 |
| Cdk2/cyclin E | 0.1 |
| Cdk7/cyclin H | 0.5 |
| Cdk9/cyclin T1 | 0.8 |
| Cdk4/cyclin D1 | 14.2 |
| ERK-2 | 1.2 |
| PKA | >50 |
| PKC | >50 |

Although the use of roscovitine as an antiproliferative agent is known in the art, to date, there has been no suggestion that it would be effective in the treatment of CLL, which is known to be particularly difficult to treat and is often resistant to conventional treatments.

Therapeutic Activity

For all embodiments of the invention, preferably the roscovitine is in the form of the R enantiomer, namely 2-(1-R-hydroxymethylpropylamino)-6-benzylamino-9-isopropyl-purine, hereinafter referred to as “CYC202”.

Chronic Lymphocytic Leukemia (CLL)

Chronic lymphocytic leukemia (CLL) is a heterogeneous group of diseases characterized by different maturation states of the B-cells and T-cells, which are related to the aggressiveness of the disorder. The disorder is characterised by clonal proliferation of immunologically immature and functionally incompetant small lymphocytes. CLL is commonly classified into separate categories, including B-cell chronic lymphocytic leukemia of classical and mixed-types, B-cell and T-cell prolymphocytic leukemia, hairy-cell leukemia and hairy-cell variant, splenic lymphoma with circulating villous lymphocytes, large granular lymphocytic leukemia, adult T-cell leukemia/lymphoma syndrome and leukemic phases of malignant lymphomas of both B-cell and T-cell types.

Treatment of CLL is generally individualized. No specific treatment is required in older patients having an indolent form of the disease. However, other patients with more advanced disease or with disease having a more rapid course may have a median survival of less than two years. Therefore, some sort of treatment should be pursued. The majority of patients have an intermediate prognosis, and although they fare reasonably well without treatment for several years, ultimately they will require some form of therapy.

To date, typical treatment for CLL has involved the administration of chlorambucil, a chemotherapeutic agent. Combination chemotherapy is generally used only in advanced cases. Radiation therapy has been effectively used, particularly if splenic enlargement is present and bone marrow transplantation has been successful with younger patients [Foon et al., Leukemia 6 (Supp. 4): 26-32, 1992]. U.S. Pat. No. 5,455,280 suggests a method for treating CLL using therapeutically effective amounts of beta-carotene. More recently, the nucleoside fludarabine, a fluorinated adenine analog, and 2-chlorodeoxyadenosine, a deoxyadenosine analog, have been found to be effective. Both analogs are resistant to deamination [Keating et al., Leukemia 6(Supp. 4): 140-141, 1992]. All of these therapies focus on elimination (with replacement, in the case of the transplants) of the malignant cells. However, CLL still remains a particularly difficult disorder to treat.

B-Cell Chronic Lymphocytic Leukemia (B-CLL)

In one preferred embodiment, the invention relates to the use of roscovitine, or a pharmaceutically acceptable salt thereof, in the treatment of B-cell chronic lymphocytic leukemia.

B-cell chronic lymphocytic leukemia (B-CLL) is the commonist leukemia in the Western world and is to date incurable. The disease course is variable, with a proportion of B-CLL tumours having poor clinical outcomes due to mutations in either the ATM or TP53 genes that operate in a common DNA damage-response pathway.

B-CLL is characterized by proliferation and accumulation of B-lymphocytes that appear morphologically mature but are biologically immature. B-CLL is typically an indolent neoplasm and survival for years can be anticipated. B-CLL typically occurs in persons over 50 years of age. This disorder accounts for 30% of leukemias in Western countries, with 10,000 new cases being diagnosed annually in the United States alone.

The characteristic phenotype of B-CLL cells involves expression of CD5, a marker diagnostic of the disease, and at least one other B-cell marker (CD19, CD20 or CD23), as well as low expression of surface immunoglobulins¹, which upon organ infiltration cause lymph-node enlargement and hepatosplenomegaly. In the advanced stages of the disease, bone marrow occupation by the abnormal lymphocytes causes bone marrow failure, resulting in anemia and thrombocytopenia.

The B-cells in CLL have receptors for mouse erythrocytes, a marker of immature B-cells. An increased number of T-cells has been reported in this disorder with an increase in the number of T-suppressor cells. Typically, an inversion of the T-helper/suppressor ratio results, with increased suppressor T-cells and decreased helper T-cells. The absolute number of natural killer cells may also be increased. Chromosome analysis provides prognostic information about overall survival, in addition to that supplied by clinical data in patients with B-CLL.

The clinical course of the disease is remarkably variable, remaining stable for extended periods in some patients while in others progress is much more rapid. The standard treatments for B-CLL include chlorambucil and more recently the purine analogue fludarabine. However, no clear increase in overall survival has been observed following the introduction of fludarabine, and relapse eventually occurs in nearly all fludarabine-treated patients.²

In a preferred embodiment of the invention, the cytotoxic effect of roscovitine is selective for B-CLL cells over normal lymphocytes.

Studies by the applicant have revealed that normal lymphocytes treated with the same concentration of CYC202 were also susceptible to the cytotoxic effects of the drug. However, these cells showed a lower cytotoxic response that was also delayed in time. Thus, CYC202 exhibits selective cytotoxicity towards B-CLL cells compared to normal B cells. Given the fact that apoptosis in B-CLL cells can be induced following a minimum of 6 hours of incubation with CYC202, manipulation of the dose and interval between the administration of CYC202 in vivo could further differentiate responses between B-CLL cells and normal lymphocytes. Indeed, in support of this notion, low toxicity of CYC202 has already been reported in a clinical setting.⁷

T-Cell Chronic Lymphocytic Leukemia

In another preferred embodiment, the invention relates to the use of roscovitine, or a pharmaceutically acceptable salt thereof, in the treatment of T-cell chronic lymphocytic leukemia.

T-Cell chronic lymphocytic leukemia (T-CLL) comprises less than 5% of all cases of CLL and consists of two entities. One variety has the immunophenotype CD3+, CD4−, CD8+, HNK-1T and is known as large granular lymphocytosis. A second form of T-CLL has the phenotype CD3+, CD4+, CD8− [Pathology, Second Edition, Emanual Rubin, John L. Farber, p 1067].

In large granular lymphocytosis, the neoplastic cells are large and have a moderate amount of cytoplasm with abundant azurophilic granules. These lymphocytes are thought to be related to the natural killer (NK) cell population. In 85% of cases, large granular lymphocytosis is an indolent and chronic disorder, whereas a small minority have an aggressive clinical disorder. The disease is characterised by a persistant increase in circulating large granular lymphocytes, splenomegaly, and neutropenia (with consequent repeated infections) and is frequently associated with rheumatoid arthritis.

CD4+ T-CLL is most common in young adult men and features a markedly elevated peripheral blood lymphocyte count. The neoplastic T helper cells are morphologically indistinguishable from B-CLL lymphocytes, although the nuclear contours are sometimes irregular or cerebriform. Skin involvement (dermatotropism) is common, and there is usually prominent hepatosplenomegaly. Infiltration of the bone marrow and central nervous system are characteristic features. CD4+ T-CLL is aggressive, and the mean survival is only 1 year.

Prolymphocytic Leukemia

In another preferred embodiment, the invention relates to the use of roscovitine, or a pharmaceutically acceptable salt thereof, in the treatment of prolymphocytic leukemia.

Prolymphocytic leukemia is a distinctive variant of B-CLL in 80% of cases and of T-CLL in 20%. Neoplastic B prolymphocytes express more abundant surface membrane immunogloblulin than B-CLL cells and appear to be immunologically immature. Prolymphocytic leukemia is characterised clinically by massive splenomegaly and by a marked elevation of the leukocyte count (greater than 50% prolymphocytes). Lymphadedenopathy is inconspicuous in B-cell prolymphocytic leukemia, whereas moderate lymphadenopathy is often observed in the T cell variety. Prolymphocytic leukemia is most common in elderly men (4:1 male predominance). It is an aggressive disease, with a mean survival of 2 to 3 years [Pathology, Second Edition, Emanual Rubin, John L. Farber, p 1067].

T-Cell Prolymphocytic Leukemia (T-PLL)

In one particularly preferred embodiment, the invention relates to the use of roscovitine, or a pharmaceutically acceptable salt thereof, in the treatment of T-cell prolymphocytic leukemia (T-PLL).

T-PLL is a rare chronic lymphoproliferative disorder affecting mature T-cells. The disease occurs at an advanced age, typically in the seventies or eighties, and has a slight male predominance. Although patients display similar initial symptoms as B-PLL, T-PLL is now recognised as a malignancy in its own right with distinct clinical and laboratory features, characterised by an insidious onset and poor outcome. T-PLL represents only 3% of mature B- and T-cell leukemias, but approximately 20% of prolymphocytic leukemias. In 20% of T-PLL cases the cells are small with an inconspicuous nucleolus that is only ascertained by electron microscopy. These cases have been designed as small cell variants of T-PLL.

T-prolymphocytes have the phenotype of mature postthymic lymphocytes: CD1a−, terminal deoxynucleotidyl transferase—TdT−, CD2+, CD3+, CD5+, CD7+. In respect of CD4 and CD8 expression the most common phenotype is CD4+/CD8−. Coexpression of CD4 and CD8 (double positive phenotype) is found in about 25% of cases.

The disease is aggressive and progresses rapidly. Clinical experience shows that the number of effective therapeutic agents in T-PLL treatment is limited. Survival rate varies from 7 months (for untreated patients) to 17.5 months (in responding to the therapy patients). To date, treatment has centred on the administration of agents such as chlorambucil, cyclophosphamide, doxorubicin and vincristine, which give partial success. Although some success has been observed with 2-deoxycoformycin and CD52 antibody (campath-1H), the therapy is still a clinical problem and a more effective therapeutic approach remains to be found.

Inhibition of CDK

In one preferred embodiment, the roscovitine is administered in an amount sufficient to inhibit at least one CDK enzyme.

Preferably, the CDK enzyme is selected from CDK1, CDK2, CDK4, CDK7 and CDK9.

In one particularly preferred embodiment, the CDK enzyme is CDK2.

In another particularly preferred embodiment, the CDK enzyme is selected from CDK7 and CDK9.

Mutant ATM and Mutant TP53 Tumours

Previous studies have shown that up to 30% of B-CLL tumours have a poor clinical outcome due to defects in the p53 pathway, involved in the induction of apoptosis following DNA damage, resulting from either mutations in the ATM gene, or the TP53 gene.^{3,4,22} Such mutations contribute significantly to drug resistance, as most current anticancer treatments exert their effects through activation of a p53-dependent apoptosis pathway. Accordingly, there is an obvious interest in novel treatments capable of bypassing this key genetic defect, i.e. there is an urgent requirement for new treatments against ATM and TP53 mutant B-CLL tumours.

In one preferred embodiment of the invention, the chronic lymphocytic leukemia is associated with mutant ATM.

More preferably, the chronic lymphocytic leukemia is B-CLL associated with mutant ATM.

In another preferred embodiment of the invention, the chronic lymphocytic leukemia is associated with mutant TP53.

More preferably, the chronic lymphocytic leukemia is B-CLL associated with mutant TP53.

Studies by the applicant investigated the in vitro activity of CYC202 against a total of 26 B-CLLs, including a subset of ATM and TP53 mutant tumours. The results were compared with the cytotoxic activity induced by ionising radiation (IR) and fludarabine.

B-CLL cells treated with CYC202 at concentration of 5 μg/ml and above exhibited high levels of apoptosis within 24 hours of treatment, irrespective of ATM or TP53 gene status. Thus, surprisingly, ATM mutant, TP53 and wild type B-CLL tumours are equivalent in their response to CYC202.

This is in contrast to fludarabine treatment, where responses were delayed and considerably lower, and included a proportion of ATM mutant tumours that appeared to be non-responsive, i.e. suggesting a marked in vivo resistance to fludarabine induced apoptosis. The results also contrast with IR induced apoptosis where both ATM and TP53 mutants exhibited a clear defect in cellular killing⁸. CYC202 is therefore capable of efficiently inducing apoptosis within 24 hours of treatment in vitro in B-CLL tumour cell samples irrespective of the integrity of the p53 pathway.

Mode Of Action

In one preferred embodiment of the invention, the roscovitine down regulates expression of an anti-apoptotic gene.

Preferably, the anti-apoptotic gene comprises at least one gene selected from the group consisting of Mcl-1, Bcl-2 and Mad3.

In another preferred embodiment of the invention, the roscovitine down regulates expression of a DNA repair gene.

Preferably, the DNA repair gene comprises PCNA or XPA.

In yet another preferred embodiment of the invention, the roscovitine down regulates expression of a gene involved in transcription regulation.

Preferably, the gene involved in transcription regulation comprises at least one gene selected from the group consisting of Pol II, eIF-2, 4e and E2F.

In one particularly preferred embodiment of the invention, the roscovitine or a pharmaceutically acceptable salt thereof, is in an amount sufficient to down-regulate the expression of Mcl-1.

One aspect of the invention relates to a method of down-regulating Mcl-1 expression in B-cell chronic lymphocytic leukemia cells, said method comprising contacting said cells with roscovitine, or a pharmaceutically acceptable salt thereof.

Another aspect of the invention relates to a method of treating B-cell chronic lymphocytic leukemia in a subject, said method comprising administering roscovitine, or a pharmaceutically acceptable salt thereof, to the subject in an amount sufficient to down-regulate the expression of Mcl-1 in said subject.

Yet another aspect of the invention relates to the use of roscovitine, or a pharmaceutically acceptable salt thereof, in the preparation of a medicament for treating B-cell chronic lymphocytic leukemia, wherein the roscovitine or a pharmaceutically acceptable salt thereof, is in an amount sufficient to down-regulate the expression of Mcl-1.

Studies by the applicant have demonstrated that the effects of CYC202 on B-CLL cells preceded those induced by fludarabine by at least 24 hours and were far more pronounced. Fludarabine is thought to induce cell death through DNA damage-induced up-regulation of p53 and activation of the p53 pathway. However, the fact that in the present study the p53 mutant B-CLL tumours were sensitive to fludarabine in vitro supports the notion that fludarabine can exhibit p53-independent killing. Indeed, Pettitt et al¹⁵ reported responses to fludarabine in B-CLLs with p53 dysfunction. Furthermore, it is possible that some of mechanisms of action of fludarabine are ATM-dependent, as it was found that four out of six tumours resistant to fludarabine in vitro were ATM mutant. In contrast, CYC202 exhibited a strong killing effect on both ATM and TP53 mutant B-CLL tumours, implying a mechanism of killing independent of both ATM and p53 functions.

It has been previously found that CYC202 demonstrates potent inhibitory effects against CDK2-cyclin E, which is required for the progression of cells to S phase.⁷ However, the primarily non-cycling nature of B-CLL cells was strongly suggestive of an additional mechanism of activity for this drug. Studies by the applicant found that a number of genes were downregulated in response to treatment with CYC202, including genes involved in transcriptional and translational regulation (RNA pol II, RNA pol III), anti-apoptosis proteins (Mcl-1, Bcl-2), as well as DNA repair proteins (XPA). Down-regulation of transcription, therefore, emerged as a likely mechanism of B-CLL killing by CYC202.

During transcription, PTEF-b (CDK9/cyclin T1) and TFIIH (CDK7/cyclin H) phosphorylate the carboxy-terminal domain (CTD) of RNA polymerase II at specific target residues¹⁶ including serine 2, and this phosphorylation occurs prior to the start of transcriptional elongation.¹⁷ It has been suggested that agents that inhibit the phosphorylation of CDK9 and CDK7 kinases as well as that of the RNA pol II CTD act as transcriptional repressors.¹⁸ Consistent with the role of CYC202 as a transcriptional repressor, a rapid reduction in phosphorylation of serine 2 of RNA pol II was observed in B-CLL cells treated with this drug. Furthermore, it was found that this modification, together with the overall down-regulation of transcription, preceded the induction of apoptosis in all B-CLL tumour cells.

One of the consequences of CYC202-mediated reduced transcription includes down-regulation of a member of the Bcl-2 family, Mcl-1. Studies by the applicant found that the reduction in the level of Mcl-1 but not Bcl-2 protein coincided with the initiation of apoptosis following CYC202 treatment. Furthermore, CYC202-induced Mcl-1 disappearance temporally preceded activation of caspases-3 and -7 suggesting that Mcl-1 down-regulation may be a crucial event for induction of apoptosis in B-CLL cells via the mitochondrial pathway. Therefore, taken together, the likely sequence of events following incubation of B-CLL cells with CYC202 would include: a) inhibition of transcription by down-regulation of both RNA pol II phosphorylation and transcription-regulating genes, b) disappearance of short-lived proteins such as Mcl-1 and possibly other pro-survival factors, c) activation of mitochondria and cytochrome c release, d) activation of effector caspases and initiation of apoptosis.

In summary, it has previously been shown that ATM and TP53-mutant B-CLL tumours are associated with a generally poorer prognosis and exhibit an absence of DNA damage-induced apoptosis in vitro, which in the case of TP53 mutant tumours appears to be a consequence of both a reduction in the apoptotic signals as well as an increase in damage-induced pro-survival responses.⁸ The studies described herein have shown that CYC202 is a potent inducer of apoptosis in B-CLL cells, including those with ATM or TP53 mutations, and acts as a repressor of transcription and survival signals. Moreover, global gene expression analysis on B-CLL cells showed a significant down-regulation of genes involved in transcriptional and translational regulation, and inhibition of apoptosis, as well as DNA repair. Furthermore, CYC202 caused inhibition of RNA polymerase II phosphorylation and led to the rapid disappearance of pro-survival factor Mcl-1, at both the mRNA and protein levels, before the induction of apoptosis.

It can therefore be concluded that CYC202 is a potent inducer of apoptosis in B-CLL cells, regardless of the functional status of the p53 pathway. In view of this, and in light of its low toxicity, it may be used as a potential therapeutic agent to improve the outcome of resistant B-CLLs and provide a significant improvement in the treatment of aggressive tumours.

Pharmaceutical Compositions

Although roscovitine, (or a pharmaceutically acceptable salt, ester or pharmaceutically acceptable solvate thereof) can be administered alone, for human therapy it will generally be administered in admixture with a pharmaceutical carrier, excipient or diluent.

A preferred embodiment of the invention therefore relates to the administration of roscovitine in combination with a pharmaceutically acceptable excipient, diluent or carrier.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The active agent of the present invention can be present in the form of a salt or an ester, in particular a pharmaceutically acceptable salt or ester.

Pharmaceutically acceptable salts of the active agent of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the active agent. The man skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

The active agent of the invention may exist in the form of different stereoisomers and/or geometric isomers, e.g. it may possess one or more asymmetric and/or geometric centres and so may exist in two or more stereoisomeric and/or geometric forms. The present invention contemplates the use of all the individual stereoisomers and geometric isomers of the agent, and mixtures thereof. The terms used in the claims encompass these forms, provided said forms retain the appropriate functional activity (though not necessarily to the same degree).

The present invention also includes all suitable isotopic variations of the active agent or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the active agent of the present invention. The terms used in the claims encompass these forms.

Polymorphs

The invention furthermore relates to various crystalline forms, polymorphic forms and (an)hydrous forms of the active agent. It is well established within the pharmaceutical industry that chemical compounds may be isolated in any of such forms by slightly varying the method of purification and or isolation form the solvents used in the synthetic preparation of such compounds.

Prodrugs

The invention further includes the active agent of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the active agent, the metabolic stability and length of action of the agent, the age, body weight, general health, sex, diet, mode and time of administration, rate of excretion, drug combination, the severity of the particular condition, and the individual undergoing therapy. Dosages and frequency of application are typically adapted to the general medical condition of the patient and to the severity of the adverse effects caused, in particular to those caused to the hematopoietic, hepatic and to the renal system. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, or from 2 to 20 mg/kg body weight. More preferably the agent may be administered at a dose of from 0.1 to 1 mg/kg body weight.

As described above, roscovitine is preferably administered in a therapeutically effective amount, preferably in the form of a pharmaceutically acceptable amount. This amount will be familiar to those skilled in the art. By way of guidance, roscovitine is typically administered orally or intravenously at a dosage of from about 0.05 to about 5 g/day, preferably from about 0.5 to about 5 g/day or 1 to about 5 g/day, and even more preferably from about 1 to about 3 g/day. Roscovitine is preferably administered orally in tablets or capsules. The total daily dose of roscovitine can be administered as a single dose or divided into separate dosages administered two, three or four times a day.

Combinations

In one preferred embodiment of the invention, roscovitine is administered in combination with one or more other antiproliferative agents. In such cases, the compounds of the invention may be administered consecutively, simultaneously or sequentially with the one or more other antiproliferative agents.

It is known in the art that many drugs are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining drugs are that it may promote additive or possible synergistic effects through biochemical interactions and also may decrease the emergence of drug resistance which would have been otherwise responsive to initial treatment with a single agent.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery.

This invention is further illustrated by the following examples which should not be construed as limiting. The contents of all references, patents and published patent applications cited throughout this application, as well as the figures, are incorporated herein by reference.

### EXAMPLES

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference

Preparation of Roscovitine

CYC202 was prepared in accordance with the method disclosed in EP0874847B (CNRS).

### Example 1

In Vitro Activity of Roscovitine Against B-CLL Cells

Experiments were undertaken to demonstrate that roscovitine is capable of overcoming defects in the p53-dependent apoptosis pathway in B-cell chronic lymphocytic leukaemia.

B-CLL remains incurable and there is an urgent requirement for novel treatments. Most current anti-cancer treatments exert their effect through activation of a p53-dependent apoptosis pathway. However, 5-10% of B-CLL tumours exhibit mutations in the TP53 gene, a further 20-25% in the ATM gene. ATM is activated by DNA double-strand breaks and activates p53 by phosphorylation. Mutations in TP53 or ATM genes lead to impaired p53-dependent apoptosis and are associated with poor clinical outcome.

The in vitro activity of roscovitine was tested against B-CLL cells. Studies have shown that roscovitine inhibits cell cycle regulating CDK1 and CDK2 and transcription regulating CDK7 and CDK9. It causes apoptosis in a number of solid tumour cell lines, it induces tumour regression in xenografts as a single agent and in combination with chemotherapy. Clinical phase I-II studies in patients with cancer are ongoing.

8 patients were ATM mutant (7 with no ATM protein expressed, 1 with residual protein) and 10 were ATM/TP53 wild-type. Roscovitine was used at a range of 1 to 25 microg/ml. Annexin V assay [Annexin V/propidium iodide staining kit, Becton Dickinson Biosciences, USA] showed that the first signs of apoptosis occurred within 8 hours of treatment. By 16 hours, a dramatic loss of viability and an increase in the proportion of B-CLL cells in early apoptosis were evident irrespective of ATM status. Cells from all patients displayed a reduction in viability of at least 75%. Little dose-dependency was observed above 5 microg/ml as the effects of roscovitine at this concentration were already dramatic. While ATM mutant and wild-type tumours showed clear differences in response to irradiation, they displayed equal responses to roscovitine. Normal lymphocytes showed a delayed and lowered toxicity in response to 5 microg/ml roscovitine after 24 hours.

To investigate the mechanism of action of roscovitine, Western blotting [Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.;] was performed for several apoptosis-related proteins. Mcl-1 [J. Biol. Chem., 274: 1801-1813, 1999; J. Cell Biol., 128(6): 1173-1187, 1995; Proc. Nat. Acad. Sci. USA, 90: 3516-3520, 1993], an antiapoptotic protein, was downregulated in cells treated with 5 microg/ml of roscovitine. Cleavage of PARP [FASEB Journal 10: 587-597, 1996; Science, 267: 1456-1462, 1995; Biochim. Biophys. Acta, 950: 147-160, 1988; J. Biol. Chem, 271(9): 4961-4965, 1996; Nature, 371: 346-347, 1994], an indicator of apoptosis, occurred after 2 hours of treatment. Interestingly, PUMA, a p53-dependent pro-apoptotic protein [Mol Cell. 2001 March; 7(3): 673-82], was also downregulated. We conclude that roscovitine is a potent inducer of apoptosis in B-CLL cells regardless of functional status of the p53 pathway.

More detailed studies are set forth below in Example 2.

### Example 2

### Materials and Methods

B-CLL Patients

Samples were obtained from patients with an age range of 52 to 93 years. In 2 patients stage Ao was diagnosed, 8 patients had stage A, 5 stage B and 2 B/C, while in 10 patients stage C disease was confirmed. Previous and current treatment of all patients together with ATM/TP53 mutation status and responses to fludarabine and CYC202 are given in Table 2.

B-CLL Cells

Samples from 26 B-CLL patients characterised for ATM or TP53 status were tested. 7 tumours were ATM mutant (6 with no ATM protein expressed, 1 with residual ATM protein) 15 tumours were ATM/TP53 wild-type, and 4 tumours were TP53 mutant.⁸ Mononuclear cells were separated by density centrifugation of whole blood obtained from B-CLL patients and frozen in a viable state in 90% Foetal Bovine Serum (FBS) and 10% Dimethyl sulphoxide (DMSO). For experiments cells were thawed, washed in pre-warmed RPMI containing 1% bovine serum albumin (BSA, Sigma, Gillingham, UK) and glutamine, and cultured for a minimum of 3 hours at a density of 1×10⁶ cells per ml. For the in vitro sensitivity tests cells were plated in RPMI-1% BSA/glutamine in 24-well plates at approximately 1×10⁶ cells per ml.

Separation of Normal B Cells

Mononuclear cells (MNCs) from normal donors were obtained by density centrifugation as described above. Whole blood was mixed with RosetteSep B cell enrichment antibody cocktail (StemCell Technologies, London, UK), incubated at room temperature for 20 minutes, diluted 1:1 with PBS-2% FBS, layered over Lymphoprep and centrifuged. B cells were washed, counted, and plated for experiments.

Induction of Apoptosis In Vitro

Drugs

CYC202 was resuspended in DMSO, filter-sterilised and frozen as aliquots at −20° C. For experiments, aliquots were thawed, diluted 1:10 in culture medium and added to wells. Fludarabine was resuspended in sterile water and kept at 4° C. For sensitivity experiments each tumour sample was divided into two aliquots, half irradiated with 5 Gy and incubated without any drug and with each of the two drugs separately and combined together (concentrations used were 20 μM fludarabine and 5, 10 and 25 μg/ml CYC202 for all samples) while the second aliquot was not irradiated but treated in the same way with and without drugs. For wash-off experiments, cells were incubated in the presence of CYC202 for different lengths of time, then were washed, replated in fresh medium and the cell viability determined at 24 and 48 hours.

Irradiation

Tumour samples were resuspended in RPMI-1% BSA and irradiated with 5 Grays (Gy), using a Precisa 217 source that emits gamma-type rays (Pantatron Ltd, Gosport, Hampshire, UK).

Apoptosis Assays

An annexin V apoptosis kit (BD Pharmingen, Oxford, UK) was used to measure apoptosis in cell populations. Cells were plated as described in “B-CLL cells” above. B-CLL and control cells were treated with drugs for 0, 4, 8, 16, 24, 48, 72, and 96 hours before being harvested and washed in cold PBS. Cell pellets were resuspended, and 100 μl of 1× buffer provided by the manufacturer was added to each tube. 5 μl of annexin V and 5 μl of propidium iodide (PI) were then added to all tubes except the controls. After lightly mixing, the tubes were stored in the dark for 15-45 minutes before addition of 500 μl of 1× buffer and analysis using a Coulter Epics XL-MCL Flow cytometer (Beckman Coulter, Calif., USA).

Western Blotting

Cells were plated in non-tissue culture-treated 6-well dishes in RPMI-1% BSA+glutamine, and allowed to recover in culture for at least 3 hours prior to the addition of CYC202. Following addition of CYC202 (or DMSO as a control), plates were lightly agitated and returned to the incubator. At the indicated time points, cells were harvested, washed in cold PBS and cell pellets snap-frozen in liquid nitrogen and stored at −80° C. Defrosted cell pellets were lysed for 30 minutes on ice in 100-150 μl of TGN buffer (50 mM HCl, 150 mM NaCl, 10% glycerol, 1% Tween-20, 0.2% NP-40 and 50 mM β-glycerophosphate) containing proteinase inhibitors (DTT, VO₄, NaF, AEBSF, aprotinin, leupeptin and pepstatin). Lysates were centrifuged for 20 minutes at 4° C. at 15,000 rpm, supernatants collected and snap-frozen in aliquots. Protein content was determined for each sample using Bradford reagent (Bio Rad, Hemel Hempstead, UK). Equal amounts of protein were run on 8, 10 or 12% acrylamide gels (Bio Rad) and subjected to standard western blotting procedures. Primary antibodies against Mcl-1 and XPA were purchased from BD Pharmingen (Oxford, UK), Bcl-2, PARP n20, and Mad3 from Santa Cruz (Autogen Bioclear, Calne, UK) and actin from Sigma-Aldrich (Dorset, UK). Anti-RNA pol II and anti-RNA pol II serine 2 were obtained from Covance Research Products (Cambridge Bioscience Ltd, Cambridge, UK). Secondary antibodies anti-mouse IgG peroxidase conjugate, and anti-goat IgG peroxidase conjugate were purchased from Sigma-Aldrich. Anti-rabbit HRP was purchased from DAKO (Ely, UK). Immobilised antigens were detected using an ECL Western blotting detection system (Amersham Biosciences, Chalfont St Giles, UK) and exposure to x-ray film (Hyperfilm™, Amersham Biosciences).

Flow Cytometry

CD5-PE (T1-RD1) and CD19-FITC (B4-FITC) antibodies were obtained from Coulter Clone (High Wycombe, UK). For staining, cells were incubated in PBS with 10% FBS for 20 minutes at room temperature, then washed, resuspended in PBS-10% FBS and stained for 30 minutes at room temperature in the dark. After incubation, cells were washed, resuspended in PBS-1% FBS, and analysed by flow cytometry using a Coulter Epics XL-MCL flow cytometer.

Microarray Analysis

Five B-CLL tumours, 2 ATM mutant and 3 ATM/TP53 wild-types, were subjected to microarray analysis before and 4 hours after incubation with 5 μg/ml CYC202. After prolonged incubation for a further 20 hours, an aliquot of each sample was stained for CD5 and CD19, and another tested for apoptosis using annexin V staining. For microarray analysis, extraction of total RNA, first and second strand cDNA synthesis, in vitro transcription and chip hybridisation were performed as previously described.⁸ After incubation, the chips were washed and labelled with streptavidin-phycoerythrin and the resulting signal amplified by a second round of staining. Washing and staining steps were performed using the fluidics station (Affymetrix, High Wycombe). Chips were scanned with a confocal argon ion laser (Agilent Technologies, Calif., USA).

Data Collection, Normalization, Filtering and Statistical Analysis

Expression values were obtained for all 10 hybridisations using Affymetrix Microarray Suite 5.0 software. Data quality was assessed using MAS5.0 report files and Genespring 5.1 (Silicon Genetics, San Carlos, USA). For analysis with GeneSpring 5.1 software, raw data was exported from MAS5.0 and values were normalised to the median signal value for each array. For comparison on the basis of drug response, additional normalization was performed to the mean level of gene expression in the untreated samples. The U95Av2 GeneChip contains 12,627 transcripts including control bacterial genes. To devise a list of informative genes, Genespring 5.1 was used to generate experimental interpretations to compare tumours before and after exposure to CYC202. In all cases, variance was determined using a global error model based on the replicates. Genes whose signal strengths did not significantly exceed background values and genes whose expression did not reach a threshold value for reliable detection (based on the Affymetrix MAS5.0 probability of detection values p≦0.1) were excluded in at least 3 samples out of 5 B-CLL replicates. Finally, genes whose levels of expression did not vary between responses to drug by more than 1.5 fold were also excluded. The remaining genes were considered to be informative and were subjected to parametric (Welch) t-testing between two conditions (untreated and treated cells) using a global error model with the variance statistic derived from replicates.

Finally, to reduce discovery of false differential gene expression, Benjamini-Hochberg multiple testing correction filtering was applied.

As an alternative method, scanned images of microarray chips were analysed using probe level quantile normalisation.⁹ Subsequently, robust multi-array analysis¹⁰ on the raw CEL files was preformed using the Affymetrix package of the Bioconductor (http://www.biocondutor.org) project. Differentially-expressed probe sets were identified using SAM^{11,12}. Finally, hierarchical clustering of genes was performed using DNA-Chip Analyzer and default settings (dChip; Wong Lab, Dept of Biostatistics, Harvard School of Public Health, Dept. of Biostatistical Science, Dana-Farber Cancer Institute; http://www.dchip.org).

The CYC202-responsive genes that were identified by both approaches were taken into further consideration. In addition, to identify CYC202-specific pro-apoptotic responses the list of CYC202-responsive genes was compared with the list of IR-responsive genes obtained from the same tumour samples.⁸

Results

CYC202 is a Potent Inducer of Apoptosis In Vitro in B-CLL Tumours

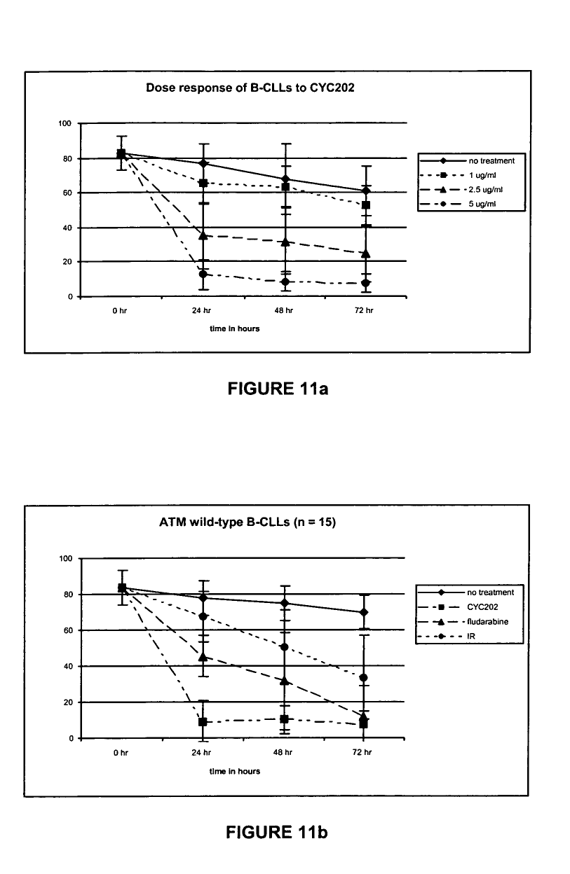

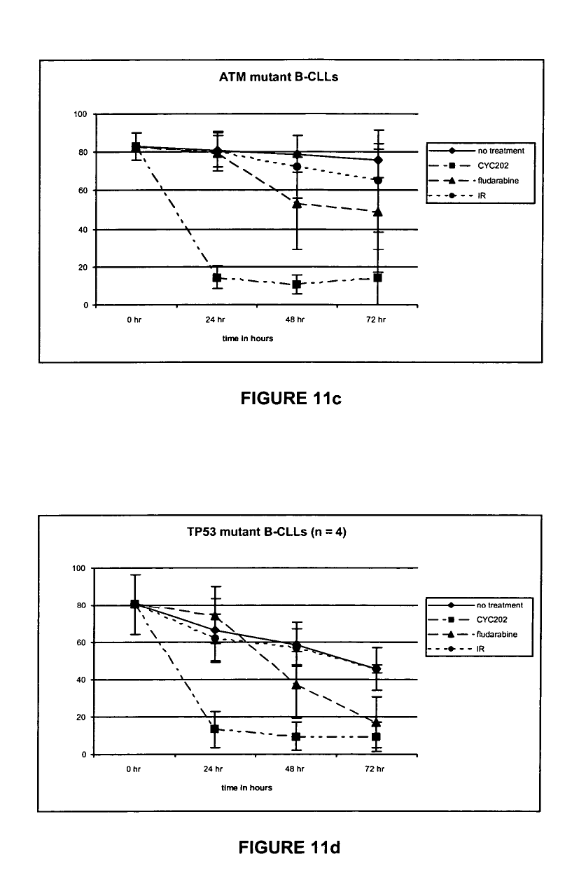

In order to establish optimal CYC202 doses and length of incubation for induction of toxicity in B-CLL cells, representative B-CLL tumour samples (1 ATM wild-type and 1 ATM mutant) were initially subjected to increasing doses of CYC202 and apoptosis assessed at 4, 8, 16, 24, 48 and 72 hours of incubation. As FIG. 11a shows, treatment at 1 μg/ml had little or no effect on cell viability or induction of apoptosis in any of the samples, whereas 2.5 μg/ml affected some but not all of the B-CLL samples (FIG. 11a). Within 24 hours of treatment with CYC202 at 5 μg/ml, however, there was a dramatic loss of viable B-CLL cells and by 48 hours of treatment most cells were in apoptosis. Little dose-dependency was observed with concentrations above 5 μg/ml (results not shown). It was also found that the first signs of apoptosis were detectable between 4 and 8 hours of treatment with 5 μg/ml of CYC202, and that by 16 hours these early effects were much more pronounced and the proportion of viable cells greatly diminished (data not shown).

To establish whether B-CLL cells treated with CYC202 for different times could escape the effects of the drug if left to recover in drug-free culture following drug exposure, wash-off experiments were performed. The cells of 3 tumours were treated with 5 μg/ml of CYC202 for 30 minutes, 1, 2, 3, 4, 6 and 8 hours. The drug was then washed off and the cells incubated for a further 24 hours. The viability of the cultures was then tested by annexin V/PI staining. Exposure to CYC202 for less than 6 hours with subsequent recovery in culture did not induce measurable cytotoxic effects but with exposure times of 6 hours and more, a large drop in viability was seen despite a recovery period after drug exposure. These results are consistent the observed appearance of early apoptotic cells as measured by annexin V in B-CLL cultures treated for a minimum of 8 hours and indicate that a minimum time of exposure to the drug is necessary to irreversibly engage the cell's apoptotic machinery. The B-CLL samples (15 ATM wild-type tumours, 7 ATM mutant tumours, and 4 TP53 mutant tumours) were also tested by annexin V assay at 24, 48, and 72 hours following incubation with CYC202 and compared the results to killing of the same tumours by ionising radiation and fludarabine. These comparative results are shown in FIG. 11b for ATM wild-type tumours, FIG. 11c for ATM mutant tumours and FIG. 11d for TP53 mutant tumours.

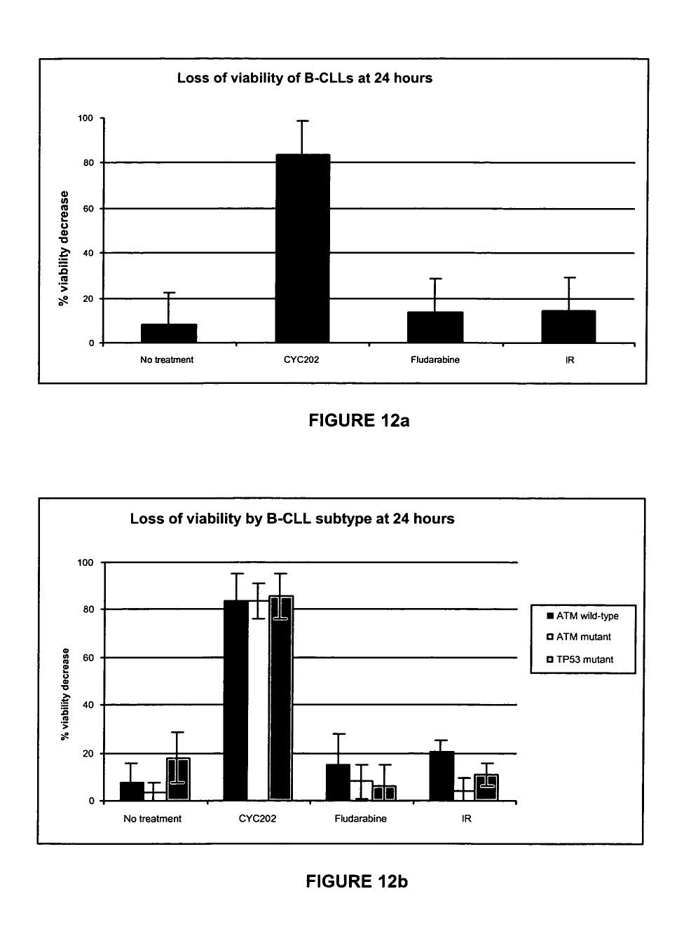

Among the 26 B-CLLs together, an average decrease in viability was observed after treatment with 5 μg/ml of CYC202 of 83.6% (range 53-97%+/−10.1%, FIG. 12a), while untreated B-CLL cells showed a decrease in viability of only 8.0%+/−8.5%. When B-CLLs were divided by genotype, the 15 ATM wild-type tumours showed an average decrease in viability of 83.5%+/−11.7% (FIG. 12b), whereas ATM mutant tumours and TP53 mutant tumours revealed a loss of viability of 83.9%+/−7.4% (FIG. 12b) and 85.8%+/−9.7% (FIG. 12b) respectively. Thus, none of the three genotypes was resistant to CYC202 at 5 μg/ml and all three groups showed a similar response to the drug.

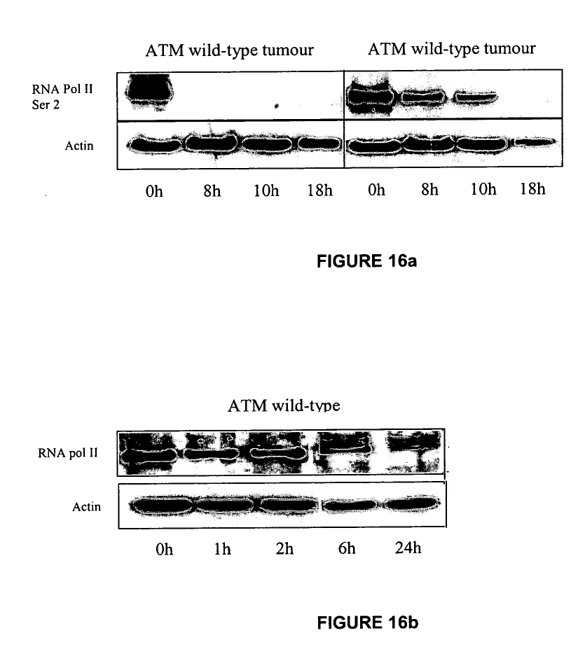

Remarkably, when compared to 5 μg/ml of CYC202, cells treated with 20 μM fludarabine showed a reduced loss of viability and reduced increase in apoptosis, regardless of subtype (FIGS. 11b, 11c and 11d). This difference was most marked in the ATM mutant and TP53 mutant subtypes where no increased loss of viability above the level of spontaneous apoptosis was observed in cells within the first 24 hours of treatment (FIGS. 11c and 11d). Fludarabine induced an overall decrease in viability after 24 hours of treatment of 13.3%+/−12.1% (FIG. 12a) for all tumour genotypes combined. For ATM wild-type tumours alone, the decrease in viability at the same time point was 14.9%+/−12.7% (FIG. 12b), compared to 7.9%+/−7.2% in ATM mutant tumours (FIG. 12b) and 16.7%+/−16.0% in 4 TP53 mutant tumours (FIG. 12b). Even after 48 hours of fludarabine treatment, decreases in viability were significantly lower than after 24 hours' incubation with CYC202. Overall, it was observed that fludarabine-induced apoptosis was much lower compared to the apoptosis induced by CYC202 for the same treatment period (summarised in Table 1 below). Interestingly, 6/23 tumours were resistant in vitro to fludarabine and 67% (4/6) of those were ATM mutants. Among fludarabine-sensitive tumours, 13 were ATM wild-type and 3 (19%) were ATM mutant. Notably, none of the 4 TP53 mutant tumour samples showed resistance to fludarabine in vitro (summarised in Table 2) although their response to the drug was slow compared to wild-type tumours.

Taken together, CYC202, but not fludarabine, was able to induce high levels of apoptosis in all B-CLL samples including those shown to be defective in irradiation-induced apoptosis. Thus, CYC202 is much more efficient at killing B-CLL cells in vitro than fludarabine, regardless of ATM/TP53 gene status.

In contrast to the effect of CYC202, and in agreement with previous observations, wild-type, ATM mutant, and TP53 mutant tumours showed little or no apoptosis 24 hours following IR (FIGS. 11c, 11d and 12b). IR-induced apoptosis become apparent 72 hours following exposure to IR, but only in tumours with ATM/TP53 wild-type sequences (FIG. 11b).

To analyse the synergism of induction of apoptosis between the three treatment modalities, 25 of the 26 tumours were subjected to combined treatments involving IR plus fludarabine, IR plus CYC202 or fludarabine plus CYC202. One tumour was treated with combinations of drugs but no irradiation. Consistent with different mechanisms of killing by fludarabine and IR, increased response rates upon irradiation were observed in 20/25 tumours treated with fludarabine over a 24-hour time period. In contrast, irradiation of CYC202-treated samples did not increase cytotoxicity, suggesting a supreme efficiency of this drug as a single agent in B-CLL killing. Similarly, the addition of fludarabine to CYC202-treated cultures showed no increase in apoptosis over the level of killing induced by CYC202 alone. It is likely, therefore, that the effects of fludarabine were masked by the much greater cytotoxicity of CYC202, especially at earlier time points.

Effect of CYC202 on Induction of Apoptosis in Normal B Lymphocytes

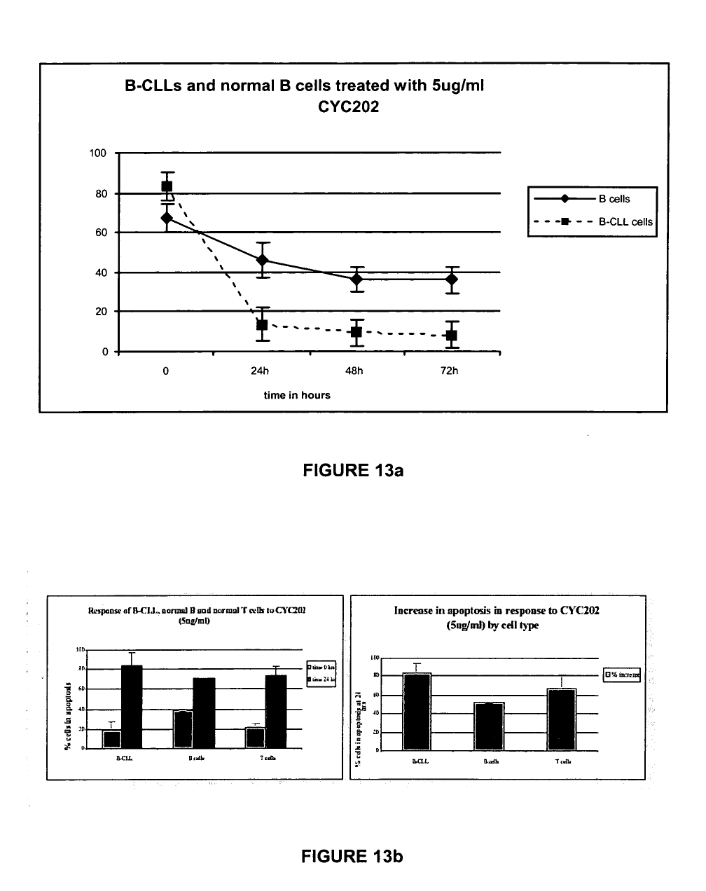

To determine the effects of CYC202 treatment on non-leukaemic B lymphocytes, cells from 5 control individuals were isolated and treated with the drug at a concentration range of 1-20 μg/ml. In contrast to the effects of CYC202 on B-CLL cells, normal B cells showed delayed and reduced toxicity in response to 5 μg/ml CYC202. Untreated B cells from control individuals showed an average decrease in viability of 4.4% (+/−1.9%) over 24 hours while the viability of B cells treated with 5 μg/ml of CYC202 for the same time dropped by 31.4%+/−17.1%. This compares to a much higher drop in viability of 83.6%+/−10.1% for B-CLL tumours treated with 5 μg/ml CYC202 for 24 hours (FIG. 13). At 48 hours, the viability of untreated B cells had dropped by 26.5%+/−19.3%, that of B cells treated with CYC202 by 47.4%+/−17.2%, and 88.3+/−8.1% for B-CLL tumours. The cytotoxicity of CYC202 against normal B cells only reached comparable levels to that observed among B-CLLs when 20 μg/ml was used. Therefore, from our data it would appear that CYC202 shows a significant degree of selective cytotoxicity toward B-CLL cells at a concentration of 5 μg/ml.

Mechanism of B-CLL Killing by CYC202

a) Effect of CYC202 on Apoptotic Pathways and Effector Caspases

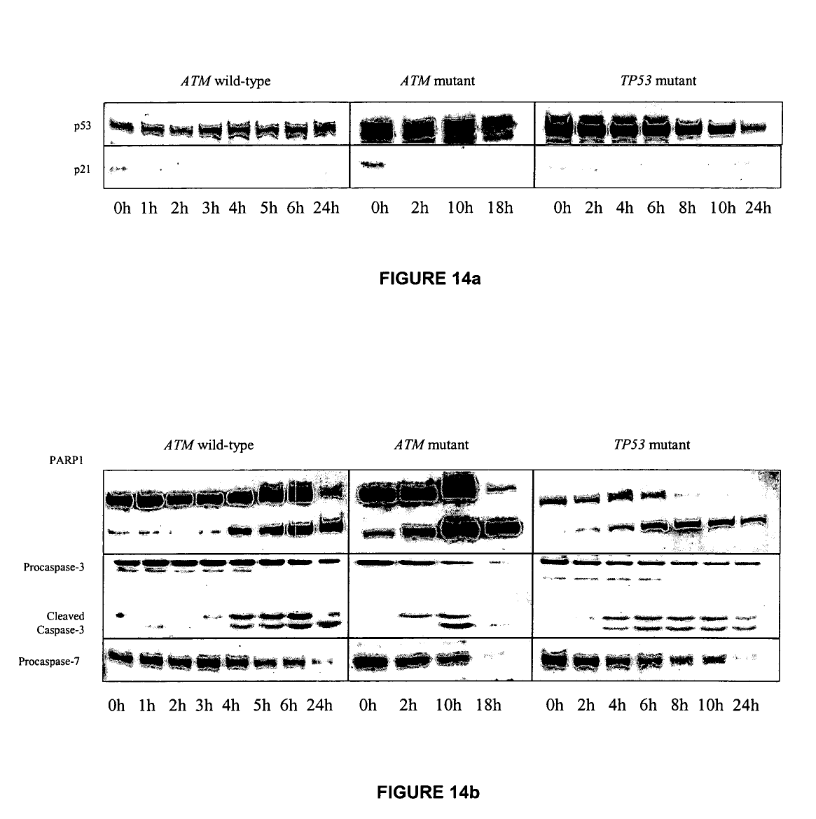

B-CLL is a tumour of slowly-cycling lymphoid cells. Given the efficient killing within 24 hours of incubation with CYC202 of all B-CLL tumours, including those with defective p53 pathways, it was plausible to reason that B-CLL killing by CYC202 involves a mechanism other than cell cycle inhibition or activation of p53-dependent transcription. Indeed, western blotting revealed an absence of p53 activation following incubation with CYC202 (FIG. 14a). Despite some increase in the levels of p53 between 3 and 6 hours of treatment with CYC202 in an ATM wild-type B-CLL, there was no evidence of up-regulation of the p53-responsive protein p21 (FIG. 14a). Similarly and as expected, there was also no evidence of CYC202-induced up-regulation of p53, nor of p21, in tumours with either ATM or TP53 mutations. If anything, both of these tumours displayed decreases in the levels of p53 protein over the time that become barely detectable by 18-24 hours of incubation with CYC202 (FIG. 14a).

Downstream apoptotic pathway activation was analysed. Consistent with induction of apoptosis, PARP1, a target for degradation of activated effector caspase-3, was cleaved by 6-24 hours of CYC202 treatment in representative tumours of all three B-CLL subtypes (FIG. 14b). Furthermore, direct caspase-3 activation was confirmed in all three B-CLL subtypes by the cleavage of procaspase-3 and the concomitant appearance of active (cleaved) caspase-3, whereas cleavage and disappearance of procaspase-7 indicated activation of caspase 7 (FIG. 14b). It can be concluded that CYC202-induced killing includes activation of apoptotic pathways downstream of p53.

b) Effect of CYC202 on Transcription

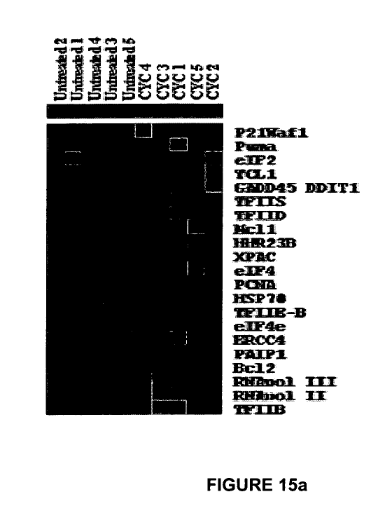

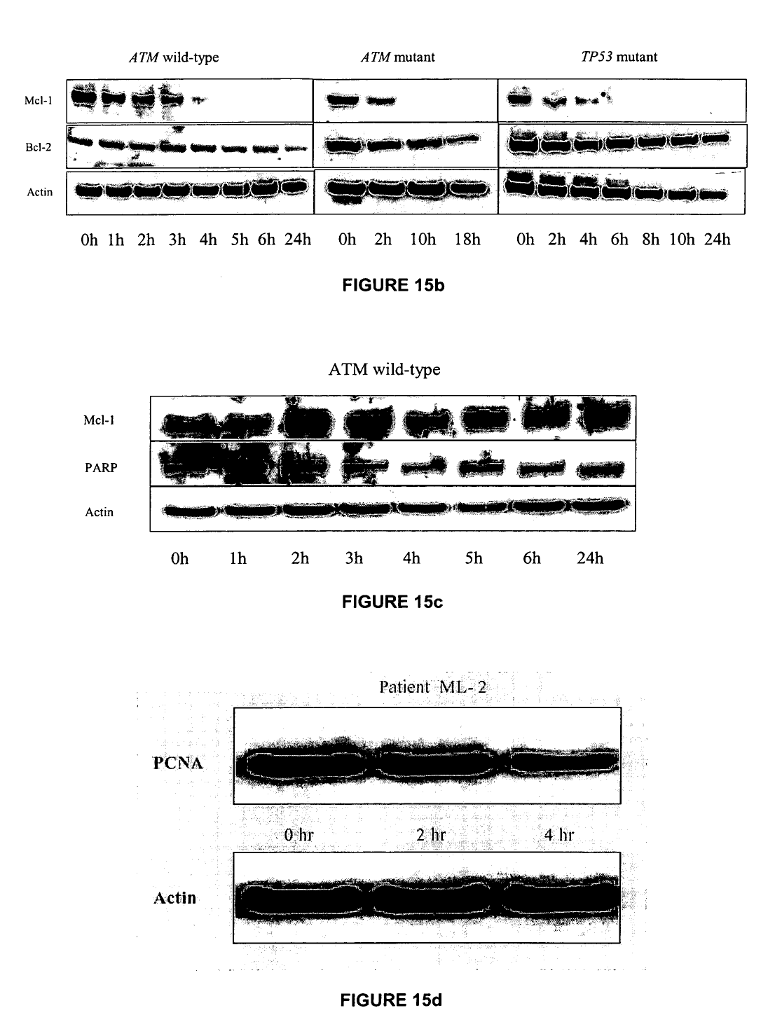

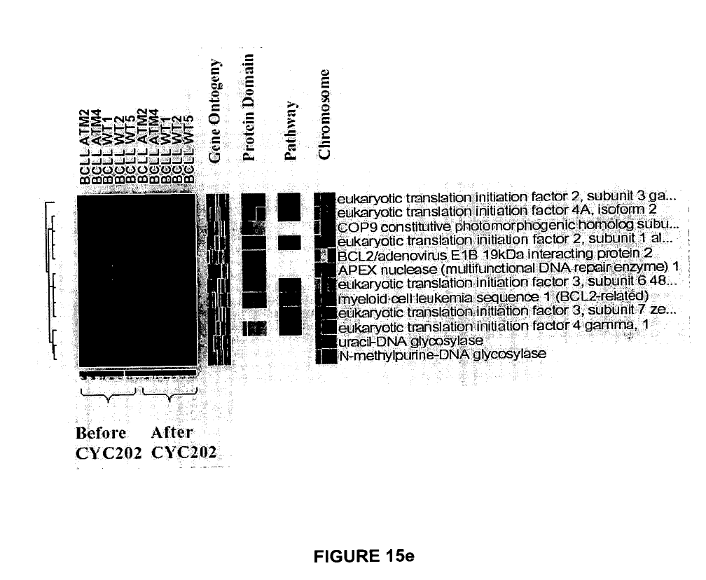

In order to investigate the impact of CYC202 on transcription in B-CLL cells, global gene expression profiling was undertaken using U95A Affymetrix microarray chips in five representative samples (3 ATM/TP53 wild-type and 2 ATM mutant) before and 4 hours after exposure to 5 μg/ml of CYC202. Cells were harvested and processed for microarray analysis as described in Materials and Methods. An aliquot of treated cells was also tested by annexin V assay analysis following 24 hours of incubation with CYC202 to confirm that apoptosis had been induced in all tumours. Gene expression results for CYC202-treated samples were compared to baseline gene expression for the corresponding untreated samples. Following filtration of uninformative genes and statistical testing, including multiple testing correction, we identified 547 genes that were downregulated more than 1.5 fold and 135 genes that were upregulated more than 1.5 fold following exposure to CYC202. While the upregulated genes pointed to diverse cellular signals, downregulated genes clearly implicated several cellular pathways that could explain the pro-apoptotic activities of CYC202 in B-CLL. First, a spectrum of genes encoding proteins involved in initiation of transcription and translation such as TFIIB, TFIID, TFIIS, TFIIE beta, RNA polymerase II and III, elongation initiation factors eiF-2 alpha, gamma and eiF-4, was clearly down-regulated after exposure to CYC202 (FIG. 15a). Furthermore, genes with anti-apoptotic properties supporting cellular survival such as Mcl-1, Bcl-2 (FIG. 15a), Mad3, NFkB subunits, several members of the heatshock family of proteins, the family of interferon cytokines and receptors were also downregulated in response to CYC202. Finally, the expression of many repair genes, including PCNA, XP-C and ERCC4, was reduced following 4 hours of exposure to the drug (FIG. 15a). Other important down-regulated pathways included MAP kinases and their downstream effectors.

The profile of CYC202 transcriptional responses was remarkably different from those previously observed following IR in the same set of B-CLL tumours.⁸ In contrast to IR-induced signals and consistent with the p53-independent nature of CYC202 transcriptional responses, CYC202 did not induce significant changes in the mRNA levels of p53-responsive genes such as p21 (FIG. 15a) and Puma in ATM/TP53 wild-type tumours. Furthermore, down-regulation of pro-survival factors Mcl-1 (FIG. 15a), heatshock proteins and NFkB genes appeared to be entirely specific to the CYC202 effect as these genes were not found to be upregulated following IR in wild-type B-CLL tumours.

Western blotting was used to confirm the differential expression of key responders to CYC202. Mcl-1 is a pro-survival gene of the Bcl-2 family important for the regulation of apoptosis in lymphoid cells.¹³ Mcl-1 expression at the protein level was investigated at various time points following incubation with CYC202 in ATM wild-type, ATM mutant and TP53 mutant tumours. An initial reduction in Mcl-1 protein levels was observed at 2 hours of incubation with 5 μg/ml of CYC202 for all tumour subtypes, followed by dramatic down-regulation and complete disappearance of the protein by 6 hours of CYC202 treatment (FIG. 15b). Interestingly, a decrease in the levels of another pro-survival protein, Bcl-2, whose mRNA was also downregulated in response to CYC202, occurred much more slowly than that for Mcl-1. Without wishing to be bound by theory, this may be a reflection of the differences in the half-lives of these proteins (0.5-3 hours for Mcl-1 vs 10-14 hrs for Bcl-2) and suggests that apoptosis may take place even in the presence of Bcl-2 protein.¹⁴

As evidence of the effect of global mRNA and protein synthesis down-regulation, both actin levels as well as the levels of proteins involved in DNA repair (PCNA and XP-C) were reduced by 24 hours. By contrast, treatment of tumours with DMSO as a control had no effect on the expression of the Mcl-1 protein nor did it induce cleavage of PARP1 (FIG. 15c) indicating that the culture conditions alone did not induce down-regulation of these proteins.

To establish the mechanism by which CYC202 globally downregulates transcription, studies were undertaken to ascertain whether CYC202 affected not only the level but also the activation of RNA polymerase II. The level of total RNA pol II protein was analysed as well as that of RNA pol II phosphorylated at Serine 2, the site associated with the elongation phase of transcription. Remarkably, it was found that the levels of phosphorylated protein were significantly reduced in B-CLL tumour samples by 8 hours of CYC202 treatment (FIG. 16a), whereas RNA pol II total protein amounts did not decrease in the same dramatic fashion (FIG. 16b). The results therefore suggest that CYC202 down-regulation of transcription may involve a direct inhibition of cyclin 9 and cyclin 7, kinases that are responsible for phosphorylation of RNA pol II protein.

### Example 3

The Effect of Roscovitine on Human T-Prolymphocytic Leukemia Cells Clinical Case Study

A 66-year-old woman was presented to the Outpatient Department because of leukocytosis discovered in a routine blood examination. Immunophenotyping performed on peripheral blood cells showed that 97% of mononuclear cells were double-positive T lymphocytes (TcR+/+/TcR+/−, CD3+, CD8+, CD4+, CD2+, CD5+ and CD7+), partially activated (CD25+, CD30−, CD38+, CD45RA−, CD45ROCD69−CD71−, HLA-DR−), expressing numerous adhesion molecules (CD11a+, CD11b+, CD11c+, CD18+, CD28+, CD62L+ and CD86−) and without expression of CD56, CD34, CD117 or B- or NK-cell markers. The cells did not express CD1a and TdT, which confirmed their mature post-thymic origin. T-cell receptor (TcR) gene analysis showed clonal TcR chain rearrangement. Surface expression of TcR+/+was detected as well. Cells were typed as human leukocyte antigen-B27 (HLA-B27) positive but no features of autoimmune disease were found.

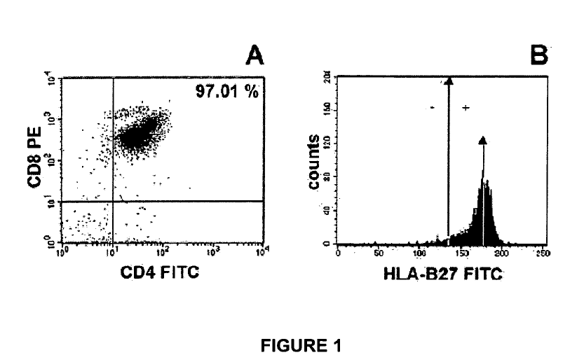

FIG. 1 shows the CD8+/CD4+ double positive immunophenotype of TPLL-1 cells (A) and HLA-B27 histogram (B). Flow cytometric analysis of periferal blood was performed on FACScan flow cytometer (Becton Dickinson, USA) with monoclonal antibodies conjugated to FITC for CD4 and phycoerythrin for CD8 (A). The left arrow in HLA-B27 histogram represents the cut-off position (B). The instrument calibration was performed accordingly using calibrating beads included in the HLA-B27 kit (Becton Dickinson Biosciensies, USA). The right arrow corresponds to the median channel value of the patient's sample, indicating HLA-B27 positivity.

The cells encoded TPLL-1 were isolated by centrifugation on a Ficoll Paque density gradient. That resulted in mononuclear cell population with over 98% of CD8+/CD4+ double positive cells of small cell morphological variant of T-PLL. Cytogenetic study was performed after stimulation with mitogens (phytohemagglutinin, pokeweed mitogen and phorbol esters) but no methaphases were obtained. Comparative genomic hybridization (CGH) analysis showed genetic gain in 8 chromosome, which is common (55%) in the cases of TPLL. Three copies of c-myc proto-oncogene without rearrangement and two copies of centromer 8 were detected by dual-color fluorescent in situ hybridization (FISH) in interphase nuclei.

Comparative Genomic Hybridisation:

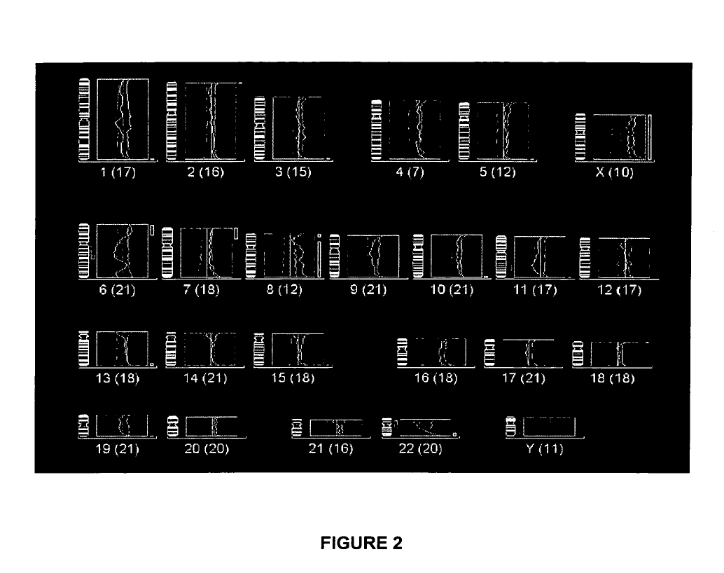

Hybridization of tumour DNA was detected with FITC and the reference DNA hybridization with TRITC (FIG. 2). Quantitation of copy number differences based on the green to ref fluorescence intensity ratio was performed by ISIS, Metasystems. Genetic loss was found at 6cen-q21, 6q26 and 11q13-q23q while genetic gain was in 6p21-p25, 7p15-p22, 8p, 8q, 22q13. These findings were in accordance with previous data [Soulier et al, Genes Chromosomes Cancer. 2001 July; 31(3): 248-54] that demonstrate high genome instablility in T-PLL.

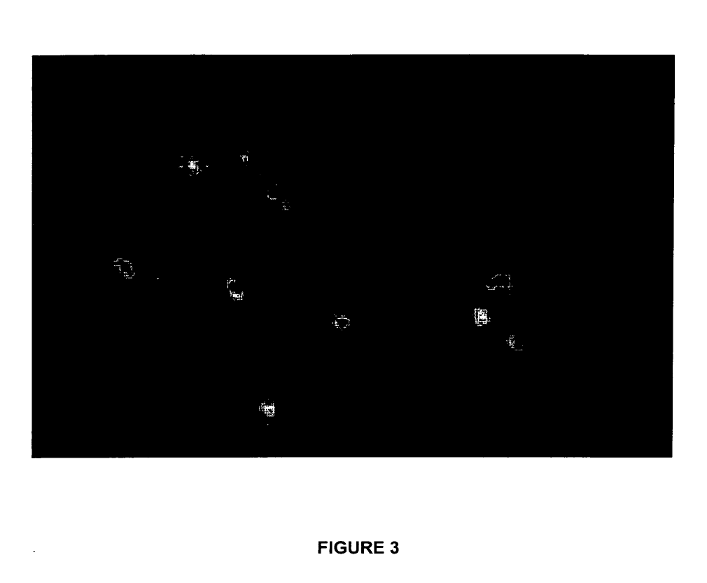

FIG. 3 shows fluorescent in situ hybridization (FISH) analysis of c-myc in TPLL-1 cells. Three sets of co-localised signals instead of two were detected indicating the presence of additional c-myc locus. Alternatively, labeled (red and green) c-myc flanking probes were applied in a FISH segregation assay. Only co-localised green and red signals are identified in all cells representing the intact c-myc loci without gene rearrangement. Hybridization signals were enumerated in 200 morphologically intact nuclei.

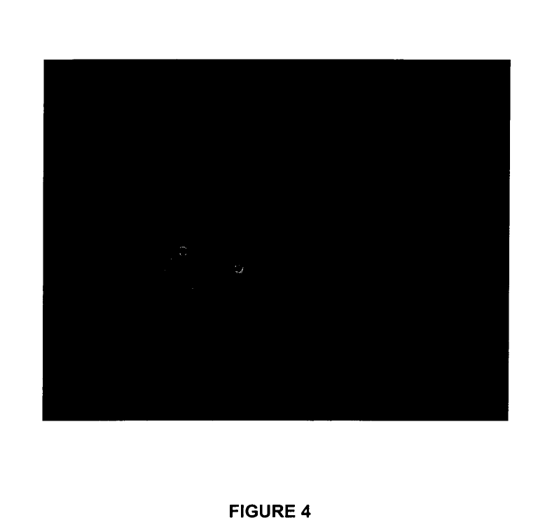

FIG. 4 shows detection of three copies of c-myc by dual colour FISH-rodamine detection of the gene (red) and FITC detection of D8Z1 chromosome 8 pericentrometric classical satellite (green).

TPLL-1 cells were treated with various kinase inhibitors (including selective for PKC isoforms, MAPK and PI-3K) but without effect on cell viability (not shown). TPLL-1 cells were incubated with 10 μM roscovitine for different time intervals and the percent of apoptotic cells was counted by FACScan.

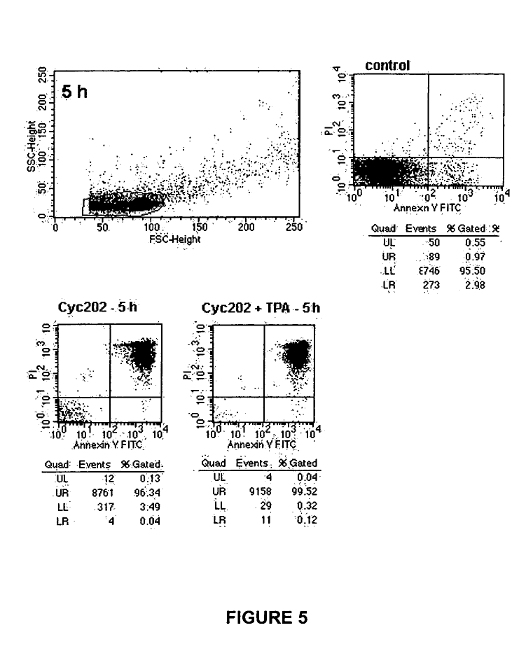

FIG. 5 shows that incubation for 5 h resulted in apoptosis of 96% of TPLL-1 cells, which was not inhibited by phorbol esters. Incubation with roscovitine for 5 h resulted in apoptosis of 96% of TPLL-1 cells, which was not inhibited by phorbol esters (10 nM TPA).

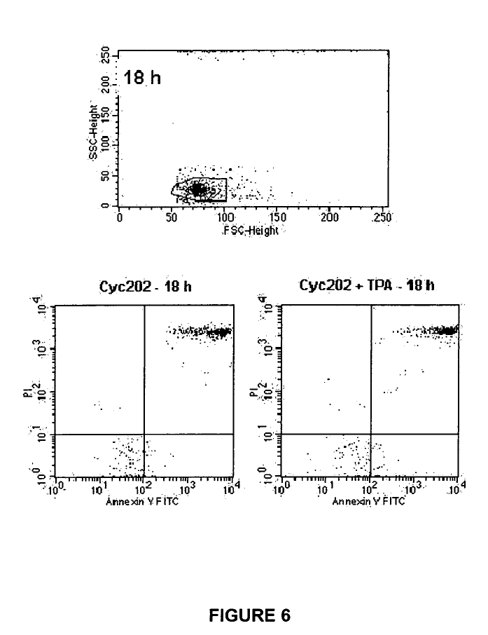

FIG. 6 shows the results of incubation for 18 h with 10 μM roscovitine. Most of the cells were lysed. The few remaining were late apoptotic cells.

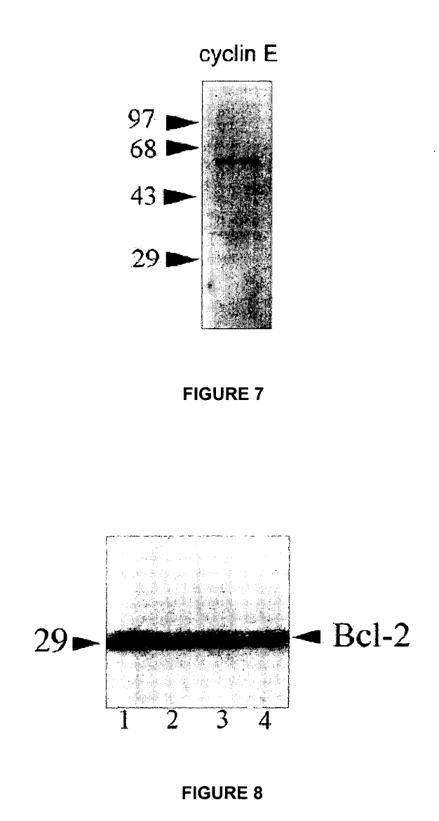

TPLL-1 cells were tested for cyclin A, B1, D1 and E expression (FIG. 7). Only cyclin E expression was detected by immunoblotting with antibody HE12 (Santa Cruz Biotechnology). Arrows indicate the position of MW marker proteins (Gibco).

FIG. 8 shows the expression of apoptotic inhibitor Bcl-2 in TPLL-1 cells. Both c-myc ASO treatment and roscovitine did not inhibit Bcl-2 expression. Expression of pro-apoptotic protein Bax was not detected (not shown).

| | |
|---|---|
| Lane 1: | control |
| Lane 2: | ASO treatment for 18 h |
| Lane 3: | 10 μM roscovitine for 20 min |
| Lane 4: | 10 μM roscovitine for 5 h |

Roscovitine induced selectively apoptosis in TPLL-1 cells. There are believed to be at least two different mechanisms of roscovitine action. First inhibition of Cdk2/CyclinE and second, activation of PI-3K pathway. Studies were undertaken to investidate the possible effect of roscovitine on stress signaling mechanisms and PI-3K pathway in TPLL-1 cells.

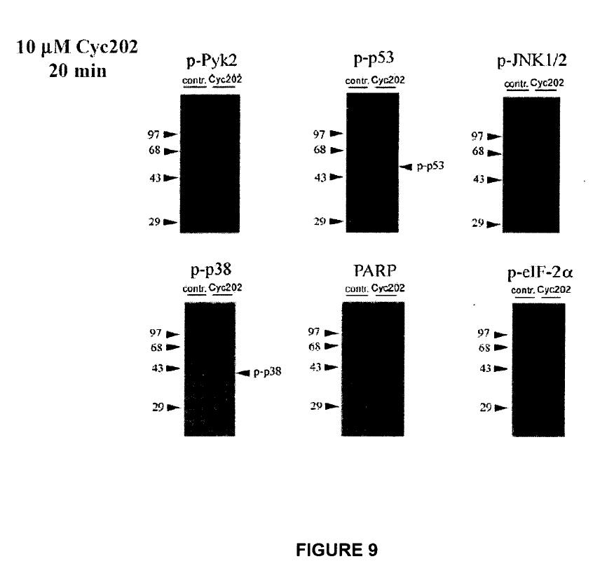

FIG. 9 shows the effect of 10 μM roscovitine on TPLL-1 intracellular stress signal pathways. The peak of p38 S51-phosphorylation was detected incubation for 20 min. Immunoblotting was performed using Stress Signal Sample Pack (BIOSOURCE Int.).

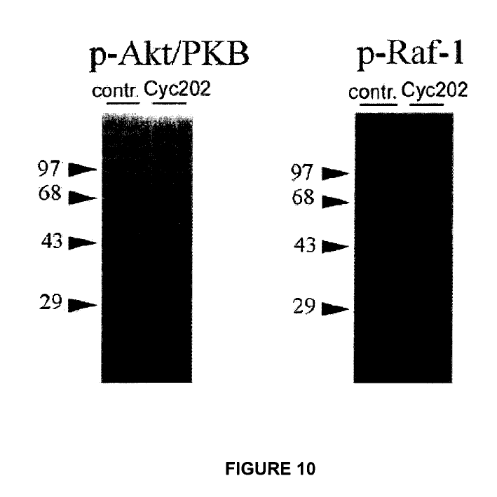

FIG. 10 shows that roscovitine did not activate PI-3K pathway in TPLL-1 cells. The results are presented of immunoblotting with antibodies specific for phosphrylated by PI-3K-dependent manner sites of the Akt/PKB (Ser-473) and Raf-1 (Ser-338). PI-3K phosphorylation at Tyr-508 after incubation with roscovitine also was not detected (not shown).

By way of conclusion, roscovitine induces apoptosis of human CD8+/CD4+ T-PLL cells. Roscovitine induces apoptosis by a PKC-independent pathway. Its effect is very rapid and selective for T-PLL cells. The possible explanation of these findings is that although T-PLL cells do not proliferate in vitro Cdk2/cyclinE activity plays a crucial role for their viability. Activation of p38 kinase by unknown mechanism could also be involved in roscovitine-induced apoptosis. Thus, roscovitine offers a new therapeutic approach in the treatment of T-PLL.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

1. Pangalis G A, Vassilakopoulos T P, Dimopoulou M N, Siakantaris M P, Kontopidou F, Angelopoulou M K. B-Chronic lymphocytic leukemia: practical aspects. Hematol Oncol. 2002; 20: 103-146.
2. Keating M J, Chiorazzi N, Messmer B, et al. Biology and treatment of chronic lymphocytic leukemia. Hematology. 2003; 153-175. American Society of Hematology Education Program Handbook. Washington D.C.
3. Pettitt A R, Sherrington P D, Stewart G, Cawley J C, Taylor A M R, Stankovic T. p53 dysfunction in B-cell chronic lymphocytic leukemia: inactivation of ATM as an alternative to TP53 mutation. Blood. 2001; 98: 814-822.
4. Stankovic T, Stewart G S, Fegan C, et al. Ataxia telangiectasia mutated-deficient B-cell chronic lymphocytic leukemia occurs in pregerminal center cells and results in defective damage response and unrepaired chromosome damage. Blood. 2002; 99: 300-309.
5. Senderowicz, A M. Small-molecule cyclin-dependent kinase modulators. Oncogene. 2003; 22: 6609-6620.
6. Fischer P M and Gianella-Borradori A. CDK inhibitors in clinical development for the treatment of cancer. Expert Opin Investig Drugs. 2003; 12: 955-970.
7. McClue S J, Blake D, Clarke R, et al. In vitro and in vivo antitumor properties of the cyclin dependent kinase inhibitor CYC202 (R-Roscovitine). Int J Cancer. 2002; 102: 463-468.
8. Stankovic T, Hubank M, Cronin D, et al. Microarray analysis reveals that TP53 and ATM mutant B-CLLs share a defect in activation of pro-apoptotic responses following DNA damage but are distinguished by major differences in activation of pro-survival responses. Blood. 2004; 103: 291-300.
9. Bolstad, B M, Irizarry, R A, Astrand, M, Speed, T P. A comparison of normalization methods for high-density oligonucleotide array data based on variance and bias. Bioinformatics. 2003; 19, 185-193.
10. Irizarry, R A, Bolstad, B M, Collin, F, Cope, L M, Hobbs, B, Speed, T P. Summaries of Affymetrix GeneChip probe level data. Nucleic Acids Res. 2003; 31, e15.
11. Tusher, V G, Tibshirani, R, and Chu, G. Significance analysis of microarrays applied to the ionizing radiation response. Proc Natl Acad Sci USA. 2001; 98: 5116-5121.
12. Storey, JD, Tibshirani, R. Statistical methods for identifying differentially expressed genes in DNA microarrays. Methods Mol. Biol. 2003; 224, 149-157.
13. Zhang B, Gojo I, Fenton R G. Myeloid cell factor-1 is a critical survival factor for multiple myeloma. Blood. 2002; 99: 1885-1893.
14. Iglesias-Serret D, Pique M, Gil J, Pons G, López J M. Transcriptional and translational control of Mcl-1 during apoptosis. Arch Biochem Biophys. 2003; 417: 141-152.
15. Pettitt A R, Sherrington P D, Cawley J C. The effect of p53 dysfunction on purine analogue cytotoxicity in chronic lymphocytic leukaemia. Br J Haematol. 1999; 106: 1049-1051.
16. Gerber M, Shilatifard A. Transcriptional elongation by RNA polymerase II and histone methylation. J Biol. Chem. 2003; 278: 26303-26306.
17. Komarnitsky P, Cho E-J, Buratowski S. Different phosphorylated forms of RNA polymerase II and associated mRNA processing factors during transcription. Genes Dev. 2000; 14: 2452-2460.
18. Lin P S, Dubois M-F, Dahmus M E. TFIIF-associating carboxyl-terminal domain phosphatase dephosphorylates phosphoserines 2 and 5 of RNA polymerase II. J Biol. Chem. 2002; 277: 45949-45956.
19. Kitada S, Zapata J M, Andreeff M, Reed J C. Protein kinase inhibitors flavopiridol and 7-hydroxy-staurosporine down-regulate antiapoptosis proteins in B-cell chronic lymphocytic leukaemia. Blood. 2000; 96: 393-397.
20. Nijhawan D, Fang M, Traer E, et al. Elimination of Mcl-1 is required for the initiation of apoptosis following ultraviolet irradiation. Genes Dev. 2003; 17: 1475-1486.
21. Cuconati A, Mukherjee C, Perez D, White E. DNA damage response and MCL-1 destruction initiate apoptosis in adenovirus-infected cells. Genes Dev. 2003; 23: 2922-2932.

22. Stankovic T, et al. Inactivation of ataxia telangiectasia mutated gene in B-cell chronic lymphocytic leukemia. Lancet. 1999 Jan. 2; 353(9146): 26-29.

TABLE 1
Effect of no treatment, CYC202 (5 μg/ml) or fludarabine (20 μM) on B-CLLs

| No of | | Decrease in viability after 24 hours | | | Decrease in viability after 48 hours | | |
|---|---|---|---|---|---|---|---|
| tumours | Genotype | No drug | CYC202 | Fludarabine | No drug | CYC202 | Fludarabine |
| 26 | All | 8.0 +/− 8.5% | 83.6 +/− 10.1% | 13.3 +/− 12.1% | 11.5 +/− 10.8% | 88.3 +/− 8.1% | 57.1 +/− 29.1% |
| 15 | ATM +/+¹ | 7.7 +/− 7.7% | 83.5 +/− 11.7% | 14.9 +/− 12.7% | 10.3 +/− 8.7% | 88.3 +/− 9.2% | 65.1 +/− 27.7% |
| 7 | ATM −/−² | 3.2 +/− 4.0% | 83.6 +/− 7.4% | 7.9 +/− 7.2% | 5.9 +/− 6.1% | 87.5 +/− 6.0% | 39.6 +/− 25.0% |
| 4 | TP53 −/−² | 17.9 +/− 12.2% | 85.8 +/− 9.7% | 16.7 +/− 16.0% | 26.1 +/− 13.7% | 89.6 +/− 8.5% | 58.1 +/− 23.9% |

¹+/+: wild-type
²−/−: mutant

TABLE 2
Gene status, drug response and clinical data of B-CLL patients

| | | | Fludarabine | CYC202 | |
|---|---|---|---|---|---|
| | ATM/TP53 | | Response | Response | |
| Patient ID | Status | Stage | in vitro | in vitro | Treatment history |
| 9206TR | ATM | A | Resistant | Sensitive | No treatment for B-CLL |
| | wild-type | | | | |
| 9348AA | ATM | A | Resistant | Sensitive | No treatment for B-CLL |
| | wild-type | | | | |
| 8375AE | ATM | C | Resistant | Sensitive | Chlorambucil in early 2001 |
| | mutant | | | | |
| 9439MS | ATM | B | Resistant | Sensitive | Chlorambucil after diagnosis of |
| | mutant | | | | stage B disease |
| 8944MK | ATM | B | Resistant | Sensitive | Fludarabine × 4, |
| | mutant | | | | Fludarabine/Chlorambucil × 2, Dead |
| 9292TT | ATM | C | Resistant | Sensitive | Fludarabine + Chlorambucil × 8, |
| | mutant | | | | PR |
| 6692MM | TP53 | C | Sensitive | Sensitive | Li Fraumeni with CLL. |
| | mutant | | | | Fludarabine, CHOP, Campath |
| 6032RB | TP53 | C | Sensitive | Sensitive | Therapy at time of sampling. |
| | mutant | | | | |
| 5266BP | TP53 | C | Sensitive | Sensitive | No treatment |
| | mutant | | | | |
| 9283PA | TP53 | Ao | Sensitive | Sensitive | No treatment |
| | mutant | | | | |
| MB | ATM | C | Sensitive | Sensitive | No treatment at time of sampling |
| | mutant | | | | |
| SS | ATM | B/C | Sensitive | Sensitive | Fludarabine (2001) |
| | wild-type | | | | |
| 8992JF | ATM | A | Sensitive | Sensitive | No treatment |
| | wild-type | | | | |
| 8998GN | ATM | B | Sensitive | Sensitive | Chlorambucil × 6 (2000) |
| | wild-type | | | | |
| 9375JM | ATM | B/C | Sensitive | Sensitive | No treatment |
| | wild-type | | | | |
| 8815DH | ATM | C | Sensitive | Sensitive | Chlorambucil in 1999 |
| | wild-type | | | | |
| 9277BL | ATM | C | Sensitive | Sensitive | No treatment |
| | mutant | | | | |
| 9355EM | ATM | C | Sensitive | Sensitive | |
| | wild-type | | | | |
| 9264JM | ATM | Ao | Sensitive | Sensitive | No treatment |
| | mutant | | | | |
| 9447TQ | ATM | A | Sensitive | Sensitive | Chlorambucil × 2 after progression |
| | wild-type | | | | to stage A (July 2002) |
| 8955ML | ATM | A | Sensitive | Sensitive | Chlorambucil June 2003 to January 2004 |
| | wild-type | | | | |
| 28SW | ATM | B | Sensitive | Sensitive | Chlorambucil × 5 |
| | wild-type | | | | |
| 102JK | ATM | A | Sensitive | Sensitive | Chlorambucil × 4, CHOP × 6 |
| | wild-type | | | | |
| 111AL | ATM | A | Sensitive | Sensitive | No treatment |
| | wild-type | | | | |
| 119BS | ATM | C | Sensitive | Sensitive | Chlorambucil |
| | wild-type | | | | |
| 9236PA | ATM | B | Sensitive | Sensitive | Chlorambucil at time of sample |
| | wild-type | | | | |

TABLE 3
Summary of gene status, drug response and
clinical data of B-CLL patients

| | Sensitivity | Sensitivity | | | |
|---|---|---|---|---|---|
| | in vitro | in vitro | | | Progressive |
| | to | to fluda- | Previously | Currently | clinical |
| Subtype | CYC202 | rabine | treated | treated | course |
| ATM/ | 15/15 | 13/15 | 2/15 | 4/15 | 3/15 |
| TP53 | | | | | |
| wild-type | | | | | |
| (n = 15) | | | | | |
| ATM | 7/7 | 3/7 | 1/7* | 2/7* | 1/7* |
| mutant | | | | | |
| (n = 7) | | | | | |
| TP53 | 4/4 | 4/4 | 3/4 | 3/4? | 3/4 |
| mutant | | | | | |
| (n = 4) | | | | | |

*= tumours resistant to fludarabine

1. A method of treating a patient suffering from chronic lymphocytic leukemia (CLL) comprising administering a therapeutically effective amount of roscovitine or a pharmaceutically effective salt thereof.

2. The method of claim 1 wherein the chronic lymphocytic leukemia is T-cell prolymphocytic leukemia (T-PLL).

3. The method of claim 1 wherein the chronic lymphocytic leukemia is B-cell chronic lymphocytic leukemia (B-CLL).

4. The method of claim 1 wherein the chronic lymphocytic leukemia is associated with mutant ATM.

5. The method of claim 1 wherein the chronic lymphocytic leukemia is associated with mutant TP53.

6. The method of claim 1 wherein the roscovitine down regulates expression of an anti-apoptotic gene.

7. The method of claim 1 wherein the anti-apoptotic gene comprises at least one gene selected from the group consisting of Mcl-1, Bcl-2 and Mad3.

8. The method of claim 1 wherein the roscovitine down regulates expression of a DNA repair gene.

9. The method of claim 8 wherein the DNA repair gene comprises PCNA or XPA.

10. The method of claim 1 wherein the roscovitine down regulates expression of a gene involved in transcription regulation.

11. The method of claim 10 wherein the gene involved in transcription regulation comprises at least one gene selected from the group consisting of Pol II, elF-2, 4e and E2F.

12. The method of claim 1 wherein the roscovitine is administered in combination with at least one additive selected from the group consisting of a pharmaceutically acceptable carrier, a diluent and an excipient.

13. The method of claim 1 wherein the roscovitine is administered in combination with at least one antiproliferative agent.

14. A method of treating a patient suffering from an ATM-mutant chronic lymphocytic leukemia comprising administering a therapeutically effective amount of roscovitine or a pharmaceutically effective salt thereof.

15. The method of claim 14 wherein the chronic lymphocytic leukemia is B-cell chronic lymphocytic leukemia (B-CLL).

16. The method of claim 14 wherein the roscovitine is administered in combination with at least one additive selected from the group consisting of a pharmaceutically acceptable carrier, a diluent and an excipient.

17. The method of claim 14 wherein the roscovitine is administered in combination with at least one antiproliferative agent.

18. A method of treating a patient suffering from a TP53 mutant chronic lymphocytic leukemia comprising administering a therapeutically effective amount of roscovitine or a pharmaceutically effective salt thereof.

19. The method of claim 18 wherein the chronic lymphocytic leukemia is B-cell chronic lymphocytic leukemia (B-CLL).

20. The method of claim 18 wherein the roscovitine is administered in combination with at least one additive selected from the group consisting of a pharmaceutically acceptable carrier, a diluent and an excipient.

21. The method of claim 18 wherein the roscovitine is administered in combination with at least one antiproliferative agent.

22. A method of treating chronic lymphocytic leukemia in a subject, the method comprising administering roscovitine, or a pharmaceutically acceptable salt thereof, to the subject in an amount sufficient to down regulate the expression of an anti-apoptotic gene in the subject.

23. The method of claim 22 wherein the chronic lymphocytic leukemia is B-cell chronic lymphocytic leukemia (B-CLL).

24. The method of claim 22 wherein the anti-apoptotic gene comprises at least one gene selected from the group consisting of Mcl-1, Bcl-2 or Mad3.

25. The method of claim 22 wherein the roscovitine is administered in combination with at least one additive selected from the group consisting of a pharmaceutically acceptable carrier, a diluent and an excipient.

26. The method of claim 22 wherein the roscovitine is administered in combination with at least one antiproliferative agent.

27. A method of treating chronic lymphocytic leukemia in a subject, the method comprising administering roscovitine or a pharmaceutically acceptable salt thereof, to the subject in an amount sufficient to down regulate the expression of a DNA repair gene in the subject.

28. The method of claim 27 wherein the chronic lymphocytic leukemia is B-cell chronic lymphocytic leukemia (B-CLL).

29. The method of claim 27 wherein the DNA repair gene comprises PCNA or XPA.

30. The method of claim 27 wherein the roscovitine is administered in combination with at least one additive selected from the group consisting of a pharmaceutically acceptable carrier, a diluent and an excipient.

31. The method of claim 27 wherein the roscovitine is administered in combination with at least one antiproliferative agent.

32. A method of treating chronic lymphocytic leukemia in a subject, the method comprising administering roscovitine, or a pharmaceutically acceptable salt thereof, to the subject in an amount sufficient to down regulate a gene involved in the regulation of transcription in the subject.

33. The method of claim 32 wherein the chronic lymphocytic leukemia is B-cell chronic lymphocytic leukemia (B-CLL).

34. The method of claim 32 wherein the gene involved in the regulation of transcription is at least one gene selected from the group consisting of RNA Pol II, elF-2, 4e and E2F.

35. The method of claim 32 wherein the roscovitine is administered in combination with at least one additive selected from the group consisting of a pharmaceutically acceptable carrier, a diluent and an excipient.

36. The method of claim 32 wherein the roscovitine is administered in combination with at least one antiproliferative agent.

37. A pharmaceutical composition for use in the treatment of chronic lymphocytic leukemia comprising roscovitine, or a pharmaceutically acceptable salt thereof, and a pharmaceutically acceptable carrier.

38. The composition of claim 37 further comprising a diluent or an excipient.
